(19) United States
(12) Patent Application Publication
(10) Pub. No.: US 2006/0014723 A1
(43) Pub. Date: Jan. 19, 2006

(54) Anti-parasitic uses of borinic acid complexes

(76) Inventors: Carolyn Bellinger-Kawahara, Redwood City, CA (US); Kirk R. Maples, San Jose, CA (US)

Correspondence Address: MORGAN, LEWIS & BOCKIUS LLP (SF), 2 PALO ALTO SQUARE, 3000 El Camino Real, Suite 700, PALO ALTO, CA, 94306, US

(21) Appl. No.: 11/153,010

(22) Filed: Jun. 14, 2005

Related U.S. Application Data
(60) Provisional application No. 60/579,476, filed on Jun. 14, 2004.

(51) Int. Cl. A61K 31/69

(52) U.S. Cl. 514/64

(57) ABSTRACT

Compositions and methods of use of borinic acid complexes, especially hydroxyquinoline, imidazole and picolinic acid derivatives as anti-parasitic agents as well as therapeutic agents for the treatment of diseases caused by parasite are described.

### 1 CROSS REFERENCE TO RELATED U.S. PATENT APPLICATIONS

This application claims the benefit under 35 U.S.C. § 119(e) of provisional U.S. Patent Application Ser. No. 60/579,476, filed Jun. 14, 2004, which is incorporated herein by reference in its entirety and for all purposes.

### 2 BACKGROUND OF THE INVENTION

2.1 Field of the Invention

The present invention relates to the field of anti-parasitic borinic acid ester compounds and uses thereof. Methods for preparing and using these compounds, and pharmaceutical compositions thereof, are also provided.

2.2 The Related Art

One hallmark of the modern era of medicine has been the decline in morbidity and mortality associated with bacterial and fungal infections. However, there is not much improvement in the filed of parasitic infections. According to the World Health Organization (WHO), an estimated 300-500 million people are infected annually by a causative parasite (most commonly Plasmodium falciparum or P. vivax, and occasionally P. malariae, P. ovale), and 1-3 million deaths per year are attributable to this disease. The significant morbidity imposed by the disease has had a deleterious effect on the economies of regions in which it is endemic and is a barrier to further economic development. Although malaria incidence leveled off briefly in the 1950's and 1960's with the advent and distribution of chemoprophylaxis and insecticides, malaria incidence has increased in the last several decades. Drug and insecticide resistance is largely responsible for this resurgence. Increased travel between industrialized nations and sub-Saharan Africa and other areas where malaria is endemic and increased prevalence of HIV-1 (which is mutually exacerbating with malaria) have worsened the problem.

Chloroquine, once the mainstay of malaria prevention and treatment, is compromised by widespread resistance in many areas where malaria is endemic, including sub-Saharan Africa, India, and much of South America. Mefloquine remains efficacious against chloroquine-resistant P. falciparum and P. vivax is associated with gastrointestinal upset, dizziness, and neuropsychological adverse effects, and is poorly tolerated. It is contraindicated in the first trimester of pregnancy, for patients with certain cardiac irregularities, and in those who are using quinine-like drugs concurrently. Atovaquone-proguanil (MALARONE™), approved by the FDA in mid 2000, is more readily tolerated, and, because of its shorter dosing regimen, elicits better compliance than do other drugs for prophylaxis. Resistance to atovaquone-proguanil has already been observed, however.

Leishmaniasis is a protozoal parasitic disease capable of causing a spectrum of clinical syndromes ranging from cutaneous ulcerations to systemic infections. The number of new cases of cutaneous leishmaniasis each year in the world is thought to be about 1.5 million, while the corresponding figure for visceral leishmaniasis is half a million. Visceral leishmaniasis is a progressive disease with mortality rate ranging from 75-95%. Treatment options are limited. The mainstays are the pentavalent antimony compounds, which were first introduced in the 1930s and have high side effects. Antifungal compounds such as amphotericin B and various azoles such as ketoconazole have been used, but these are not as effective as the pentavalent antimony compounds. Miltefosine, a phosphocholine analogue, is an experimental drug currently in Phase 2 trials.

African human trypanosomiasis (African sleeping sickness) is caused by infection with protozoan parasites of the genus trypanosome. Two main subspecies are Trypanosoma brucei rhodesiense and T. brucei gambiense. The disease is known for its potential for devastating epidemics and its fatal outcome if left untreated. Current treatment for trypanosomiasis can cause patients a number of problems, since the drugs used can have serious side effects. In late stages of the disease compounds containing arsenic must be used and these cause death in 5-10% of patients. American trypanosomiasis (Chagas' disease) is caused by an infection with the parasites T. cruzi. Chagas' disease is the leading cause of heart disease affecting an estimated 16-18 million people throughout Latin America. Current therapies are also highly toxic and only cure about 60% of patients. In some regions T. cruzi appears to be resistant to the commonly used medications.

Giardiasis is a diarrheal disease caused by Giardia intestinalis, a one-celled parasite that lives in the intestine of humans and animals. During the past twenty years, giardiasis has become recognized as one of the most common causes of waterborne disease in humans in the US and throughout the world. In adults, giardiasis is commonly treated with metronidazole; and for children under five years old with furazolidone. However, these drugs have side effects similar to the disease. Another parasitic disease is toxoplasmosis caused by Toxoplasma gondii. This one-celled parasite infects birds and mammals, including humans, worldwide. Although toxoplasmosis is not dangerous to most people, it can be life threatening to a fetus and a person with a severely weakened immune system. People with lymphoma, HIV, or who have had organ transplants, can develop life-threatening infections of the brain, heart, eye, or lungs. A combination of sulfadiazine and pyrimethamine, sometimes alternating with spiramycin, is the only known treatment for fetal toxoplasmosis during pregnancy. The treatment does not guarantee a cure of fetal toxoplasma infection, but it may reduce the risk and severity of brain and eye damage.

Amebiasis is a disease caused by a one celled parasite called Entamoeba histolytica. Amebic dysentery is a severe form of amebiasis associated with stomach pain, bloody stools, and fever. Rarely, E. histolytica invades the liver and forms abscesses. About 480 million people in the world carry amoebas in their intestine, but only about 50 million have symptoms of amebiasis and are treated with antibiotics. Cryptosporidiosis is a diarrheal disease caused by a parasite called Cryptosporidium parvum. The parasite is found in every region of the US and throughout the world. In people with AIDS, and in others whose immune system is weakened, cryptosporidiosis can be serious, long lasting, and sometimes fatal. However, there is no consistently effective treatment.

Thus, there continues to be a need in the medical arts for novel, more effective, anti-parasitic compounds, especially for treating infections that are resistant to currently available therapies.

### 3 SUMMARY OF THE INVENTION

In one aspect, the present invention describes anti-parasitic boric acid ester compounds. The compounds are borinate derivatives, especially borinic acid complexes, and include such compounds as derivatives of hydroxyquinolines, picolinic acids and imidazoles.

The anti-parasitic boron compounds of the invention are also provided as pharmaceutical compositions that can be administered to an animal, most preferably a human, for treatment of a disease having a parasitic etiology, or an opportunistic infection with a parasite in an animal, most preferably a human, in an immunologically compromised or debilitated state of health.

In preferred embodiments, the anti-parasitic borinic acid ester compounds useful in the methods and compositions of the present invention have the structures given below with preferred substituents as disclosed herein.

The invention also provides methods for preparing the anti-parasitic compounds and pharmaceutical compositions thereof, and methods of using said compounds therapeutically. Kits and packaged embodiments of the compounds and pharmaceutical compositions for the treatment of parasitic infections are also provided.

The invention also relates to methods of treating infections, preferably parasitic infections such as malaria, African human trypanosomiasis, American trypanosomiasis, leishmaniasis, giardiasis, toxoplasmosis, amebiasis and cryptosporidiosis, using the compounds, compositions, and methods provided herein.

These and other aspects and advantages will become apparent when the Description below is read.

### 4 DESCRIPTION OF SOME EMBODIMENTS OF THE INVENTION

This invention provides anti-parasitic agents and methods of use of anti-parasitic boron compounds, useful in treating and/or preventing infections caused by parasites.

The borinic acid ester compounds useful in the methods and compositions of the present invention have the structural Formulas 1 and 2: 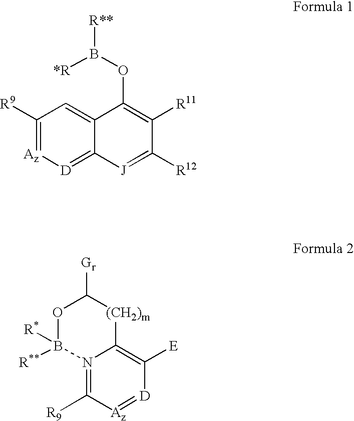
wherein: B is boron, O is oxygen;
wherein R* and R** are each independently selected from optionally substituted alkyl (C₁-C₆), optionally substituted cycloalkyl (C₃-C₇), optionally substituted alkenyl, optionally substituted alkynyl, aralkyl, optionally substituted aryl, optionally substituted heteroaryl and optionally substituted heterocyclic;
and wherein z is zero or one and when z is one, A is CH, CR¹⁰ or N;
and wherein D is N, CH, or CR¹⁴;
and wherein E is hydrogen, —OH, alkoxy, 2-(morpholinyl)ethoxy, —CO₂H, —CO₂alkyl, alkyl, —(CH₂)ₙOH (n=1 to 3), —CH₂NH₂, —CH₂NHalkyl, —CH₂N(alkyl)₂, halogen, —CHO, —CH═NOH, amino, or —CF₃;
and wherein m is zero to two;
and wherein r is 1 or 2, and wherein when r is 1, G is ═O (double-bonded oxygen) and when r is 2, each G is independently hydrogen, methyl, ethyl or propyl;
wherein R¹⁴ is selected from —(CH₂)ₖOH (where k=1, 2 or 3), —CH₂NH₂, —CH₂NH-alkyl, —CH₂N(alkyl)₂, —CO₂H, —CO₂alkyl, —CONH₂, —OH, alkoxy, aryloxy, —SH, —S-alkyl, —S-aryl, —SO₂alkyl, —SO₂N(alkyl)₂, —SO₂NHalkyl, —SO₂NH₂, —SO₃H, —SCF₃, —CN, halogen, —CF₃, —NO₂, amino, substituted amino, —NHSO₂alkyl and —CONH₂;
and wherein J is CR¹⁰ or N;
and wherein R⁹, R¹⁰, R¹¹ and R¹² are each independently selected from the group consisting of hydrogen, alkyl, cycloalkyl, —(CH₂)ₙOH (n=1 to 3), —CH₂NH₂, —CH₂NHalkyl, —CH₂N(alkyl)₂, halogen, —CHO, —CH═NOH, —CO₂H, —CO₂-alkyl, —S-alkyl, —SO₂-alkyl, —S-aryl, amino, alkoxy, —CF₃, —SCF₃, —NO₂, —SO₃H and —OH;
including salts thereof, especially all pharmaceutically acceptable salts, hydrates, or solvates.

In a preferred embodiment of either of Formulas 1 or 2, R* and/or R** are the same or are different and one of R* and R** is an optionally substituted alkyl (C₁-C₆) or R* and R** are each an optionally substituted alkyl (C₁-C₆).

In a preferred embodiment of either of Formulas 1 or 2, R* and/or R** are the same or are different and one of R* and R** is an optionally substituted cycloalkyl (C₃-C₇) or R* and R** are each an optionally substituted cycloalkyl (C₃-C₇).

In a preferred embodiment of either of Formulas 1 or 2, R* and/or R** are the same or are different and one of R* and R** is an optionally substituted alkenyl or R* and R** are each an optionally substituted alkenyl. In a further preferred embodiment thereof, the alkenyl is an optionally substituted vinyl group having the following structure: 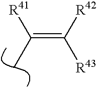
wherein R⁴¹, R⁴², and R⁴³ are each independently selected from the group consisting of hydrogen, alkyl, aryl, cycloalkyl, substituted aryl, aralkyl, —(CH₂)ₖOH (where k=1, 2 or 3), —CH₂SO₂alkyl, —CH₂NH₂, —CH₂NH-alkyl, —CH₂N(alkyl)₂, —CO₂H, —CO₂alkyl, —CONH₂, —S-alkyl, —S-aryl, —SO₂alkyl, —SO₂N(alkyl)₂, —SO₂NHalkyl, —SO₂NH₂, —SO₃H, —SCF₃, —CN, halogen, CF₃ and NO₂.

In a preferred embodiment, the methods of the invention utilize compounds of Formulas 1 or 2 wherein R* and R** are the same or are different and wherein one of R* and R** is an optionally substituted alkynyl or R* and R** are each an optionally substituted alkynyl. In a further preferred embodiment thereof, the alkynyl has the following structure: 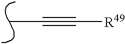
wherein R⁴⁹ is selected from the group consisting of hydrogen, alkyl, cycloalkyl, aryl, substituted aryl, aralkyl, —(CH₂)ₖOH (where k=1, 2 or 3), —CH₂NH₂, —CH₂NH-alkyl, —CH₂N(alkyl)₂, —CO₂H, —CO₂alkyl, —CONH₂, —S-alkyl, —S-aryl, —SO₂alkyl, —SO₂N(alkyl)₂, —SO₂NHalkyl, —SO₂NH₂, —SO₃H, —SCF₃, —CN, halogen, —CF₃ and —NO₂.

In a preferred embodiment of either of Formulas 1 or 2, R* and/or R** are the same or are different and one of R* and R** is an optionally substituted aryl or R* and R** are each an optionally substituted aryl. In a further preferred embodiment thereof, the aryl is phenyl having the following structure: 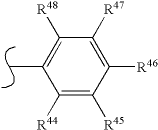
wherein R⁴⁴, R⁴⁵, R⁴⁶, R⁴⁷ and R⁴⁸ are each independently selected from the group consisting of hydrogen, alkyl, cycloalkyl, aryl, substituted aryl, aralkyl, —(CH₂)ₖOH (where k=1, 2 or 3), —CH₂NH₂, —CH₂NH-alkyl, —CH₂N(alkyl)₂, —CO₂H, —CO₂alkyl, —CONH₂, —CONHalkyl, —CON(alkyl)₂, —OH, alkoxy, aryloxy, —SH, —S-alkyl, —S-aryl, —SO₂alkyl, —SO₂N(alkyl)₂, —SO₂NHalkyl, —SO₂NH₂, —SO₃H, —SCF₃, —CN, halogen, —CF₃, —NO₂, amino, substituted amino, —NHSO₂alkyl, —OCH₂CH₂NH₂, —OCH₂CH₂NHalkyl, —OCH₂CH₂N(alkyl)₂, oxazolidin-2-yl, or alkyl substituted oxazolidin-2-yl.

In a preferred embodiment the methods of the invention utilize compounds of Formula 1 or 2 wherein R* and R** are the same or are different and wherein one of R* and R** is an optionally substituted benzyl or R* and R** are each an optionally substituted benzyl. In a further preferred embodiment thereof, the benzyl has the following structure: 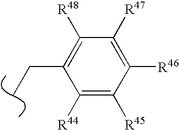
wherein R⁴⁴, R⁴⁵, R⁴⁶, R⁴⁷ and R⁴⁸ are as defined above.

In a preferred embodiment, the methods of the invention utilize compounds of Formula 1 wherein R* and R** are the same or are different and wherein one of R* and R** is an optionally substituted heteroaryl or R* and R** are each an optionally substituted heteroaryl. In a further preferred embodiment thereof, the heteroaryl has the following structure: 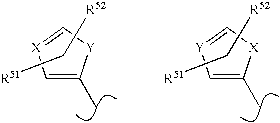
wherein X is CH═CH, N═CH, NR⁵³ (wherein R⁵³=H, alkyl, aryl or aralkyl), O, or S;
and wherein Y is CH or N when X is O, N or S;
and wherein R⁵¹ and R⁵² are each independently selected from the group consisting of hydrogen, alkyl, cycloalkyl, aryl, substituted aryl, aralkyl, —(CH₂)ₖOH (where k=1, 2 or 3), —CH₂NH₂, —CH₂NH-alkyl, —CH₂N(alkyl)₂, —CO₂H, —CO₂alkyl, —COSO₂-alkyl, —S-alkyl, —S-aryl, —SO₂alkyl, —SO₂N(alkyl)₂, —SO₂NHalkyl, —NHSO₂-alkyl, —SO₃H, —SCF₃, —CN, halogen, —CF₃, —NO₂, oxazolidin-2-yl, or alkyl substituted oxazolidin-2-yl.

In another aspect, the present invention provides methods for treating a parasitic infection in an animal, which methods comprise administering to such an animal a therapeutically effective amount of a compound having the structure shown as Formula 3: 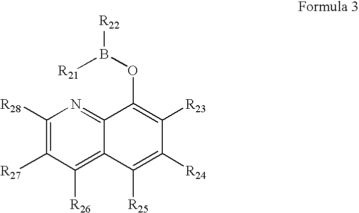
or its pharmaceutically acceptable salts, hydrates, or solvates;
wherein B is boron and O is oxygen;
R₂₁ and R₂₂ are selected independently from the group consisting of optionally substituted alkenyl, optionally substituted cycloalkyl, optionally substituted aryl, optionally substituted heteroaryl, and optionally substituted heterocyclic;
R₂₃-R₂₈ are selected independently from the group consisting of hydrogen, hydroxy, alkyl, alkoxy, halo, cyano, aryl, aralkyl, heteroaralkyl, heteroaryl, aryloxy, heteroaryloxy, heterocycyloxy, thio, alkylthio, arylthio, heteroarylthio, cycloalkyl, heterocycyl, cycloalkyloxy, formyl, carboxy, thioformyl, thiocarboxy, sulfonyl, alkylsulfonyl, arylsulfonyl, heteroarylsulfonyl, alkylsulfinyl, arylsulfinyl, heteroarylsulfinyl, amino, alkylamino, dialkylamino, arylamino, alkylsulfonylamino, arylsulfonylamino, and diarylamino. In addition, each of the above-recited alkyl-, aryl-, and heteroaryl-containing moieties is optionally substituted.

In one embodiment, the methods of the invention include administering those compounds of Formula 3 for which R₂₁ is optionally substituted alkenyl, and, more particularly, those for which R₂₁ is optionally substituted vinyl. Still more particular embodiments are those in which compounds of Formula 3 for which R₂₁ is optionally substituted vinyl and R₂₂ is optionally substituted aryl, optionally substituted heteroaryl, or optionally substituted heterocyclic are administered to an animal in need of treatment.

More particular embodiments of the method of the invention include those compounds of Formula 3 for which R₂₁ is optionally substituted vinyl and R₂₂ is optionally substituted aryl, still more particularly, R₂₂ is phenyl substituted with at least one moiety selected from the group consisting of: cyano, halo, optionally substituted heteroaryl, and optionally substituted heterocyclic. In still more particular embodiments, the moiety is selected from the group consisting of: cyano, fluoro, chloro, 4,4-dimethyl-4,5-dihydrooxazol-2-yl, and 4,5-dihydrooxazol-2-yl.

Other particular embodiments are those in which the compounds of Formula 3 for which R₂₁ is optionally substituted vinyl and R₂₂ is phenyl substituted with at least one moiety selected from the group consisting of: cyano, fluoro, chloro, 4,4-dimethyl-4,5-dihydrooxazol-2-yl, and 4,5-dihydrooxazol-2-yl and further where R₂₃-R₂₈ are selected independently from the group consisting of: hydrogen, hydroxy, alkoxy, thio, alkylthio, halo, alkyl, and cyano, more particularly those embodiments where R₂₃-R₂₇ are hydrogen, and, still more particularly, those embodiments where R₂₃-R₂₇ are hydrogen and R₂₈ is hydroxy.

In other embodiments of the methods of the invention, compounds of Formula 3 are administered in which R₂₂ is optionally substituted heteroaryl, more particularly where R₂₂ is optionally substituted pyridyl, and still more particularly where R₂₂ is 3-pyridyl. Of the embodiments just described in which R₂₂ is optionally substituted pyridyl, those in which R₂₃-R₂₈ are selected independently from the group consisting of: hydrogen, hydroxy, alkoxy, thio, alkylthio, halo, alkyl and cyano, more particularly those in which R₂₃-R₂₇ are hydrogen, and yet more specifically, those in which R₂₃-R₂₇ are hydrogen and R₂₈ is hydroxy are useful.

Still other embodiments of the method of the invention include those compounds of Formula 3 in which R₂₁ is optionally substituted cycloalkyl, and, more particularly, where R₂₁ is optionally substituted cyclopropyl. Of the latter compounds, those for which R₂₂ is optionally substituted aryl, and more specifically, where R₂₂ is optionally substituted phenyl are useful. Of those compounds where R₂₁ is optionally substituted cyclopropyl and R₂₂ is optionally substituted phenyl, compounds among those in which R₂₂ is phenyl substituted with at least one moiety selected from the group consisting of: cyano, halo, optionally substituted heterocyclic, and optionally substituted heteroaryl, more particularly where the moiety is selected from the group consisting of: cyano, fluoro, chloro, 4,4-dimethyl-4,5-dihydrooxazol-2-yl, and 4,5-dihydrooxazol-2-yl have useful properties. Among these latter combinations of R₂₁ and R₂₂, useful compounds include those for which R₂₃-R₂₈ are selected independently from the group consisting of: hydrogen, hydroxy, alkoxy, thio, alkylthio, halo, alkyl, and cyano, more particularly where R₂₃-R₂₇ are hydrogen, and still more particularly where R₂₃-R₂₇ are hydrogen and R₂₈ is hydroxy.

Other embodiments of the method of the invention include those compounds of Formula 3 in which both R₂₁ and R₂₂ independently are optionally substituted aryl, and, more specifically, where both R₂₁ and R₂₂ independently are optionally substituted phenyl. Among the latter embodiments, those for which R₂₁ and R₂₂ independently are phenyl optionally substituted with at least one moiety selected from the group consisting of: halo, alkyl, alkoxy, cyano, and cycloheteroalkyl include compounds having particularly useful properties. Within this latter set of compounds, those in which R₂₃-R₂₈ are selected independently from the group consisting of: hydrogen, hydroxy, alkoxy, thio, alkylthio, halo, alkyl, and cyano, and more particularly where R₂₈ is hydroxy; and still more particularly where R₂₈ is hydroxy and R₂₃-R₂₇ are hydrogen, or where R₂₈ is hydroxy, R₂₅ is cyano and R₂₃, R₂₄, R₂₆, and R₂₇ are hydrogen, include useful compounds.

In another aspect, the method of the invention includes administering compounds having the following structure (Formula 4): 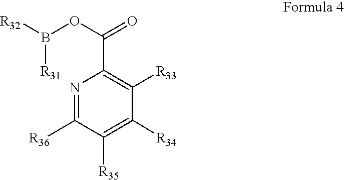
or its pharmaceutically acceptable salts, hydrates, or solvates;
wherein R₃₁ and R₃₂ are selected independently from the group consisting of optionally substituted alkyl, optionally substituted aryl, aralkyl, and optionally substituted heteroaryl. R₃₃-R₃₆ are selected from the group consisting of: hydrogen, arylcarbonyl, alkylcarbonyloxy, hydroxy, alkoxy, amino, dialkylamino, diarylamino, alkylamino, arylamino, alkylsulfonylamino, arylsulfonylamino, carboxyalkyloxy, heterocycyloxy, carboxy, hydroxyalkyl, aminoalkyl, (alkylamino)alkyl, (dialkylamino)alkyl, alkyloxycarbonyl, carbamoyl, hydroxy, alkoxy, aryloxy, thio, alkylthio, arylthio, alkylsulfonyl, dialkylsulfamoyl, alkylsulfamoyl, sulfamoyl, sulfonyl, cyano, halo, nitro, alkylcarbamoyl alkylsulfinyl, arylsulfinyl, alkanoylamino, alkyl, sulfamoyloxy wherein each of the above-recited alkyl-, aryl-, and heteroaryl-containing moieties is optionally substituted.

R₃₅ and R₃₆ together with the ring atoms to which they are attached form an optionally substituted aromatic ring.

More particular embodiments include those compounds of Formula 4 in which one of R₃₁ and R₃₂ is optionally substituted aryl. More specific embodiments are those of Formula 4 in which one of R₃₁ and R₃₂ is optionally substituted aryl and one of R₃₁ and R₃₂ is optionally substituted heteroaryl. Included among those compounds of Formula 4 in which one of R₃₁ and R₃₂ is optionally substituted aryl and one of R₃₁ and R₃₂ is optionally substituted heteroaryl are those where one of R₃₁ and R₃₂ is optionally substituted aryl and one of R₃₁ and R₃₂ is optionally substituted heteroaryl wherein the optionally substituted heteroaryl is optionally substituted pyridyl. Still more specific embodiments are those compounds of Formula 4 where one of R₃₁ and R₃₂ is optionally substituted heteroaryl wherein the optionally substituted heteroaryl is optionally substituted pyridyl and one of R₃₁ and R₃₂ is optionally substituted phenyl.

Yet more particular embodiments of the invention are those having the last recited substitution pattern and where the optionally substituted phenyl is phenyl substituted by a moiety selected from the group consisting of: alkyl, cycloalkyl, aryl, substituted aryl, aralkyl, —(CH₂)ₖOH (where k=1, 2 or 3), —CH₂NH₂, —CH₂NH-alkyl, —CH₂N(alkyl)₂, —CO₂H, —CO₂alkyl, —CONH₂, —CONHalkyl, —CON(alkyl)₂, —OH, alkoxy, aryloxy, —SH, —S-alkyl, —S-aryl, —S(O)alkyl, —S(O)aryl, —SO₂alkyl, —SO₂N(alkyl)₂, —SO₂NHalkyl, —SO₂NH₂, —SO₃H, —SCF₃, —CN, halogen, —CF₃, NO₂, amino, substituted amino, —NHSO₂alkyl, —OCH₂CH₂NH₂, —OCH₂CH₂NHalkyl, —OCH₂CH₂N(alkyl)₂, oxazolidin-2-yl, and alkyl substituted oxazolidin-2-yl.

Still other embodiments of the invention utilize compounds of Formula 4 in which both R₃₁ and R₃₂ are optionally substituted aryl, and, more particularly, where both of R₃₁ and R₃₂ is optionally substituted phenyl. Among those compounds of Formula 4 in which both of R₃₁ and R₃₂ is optionally substituted phenyl are those where R₃₃ is hydrogen, hydroxy, alkoxy, or carboxy. In more specific embodiments having this substituent pattern, the optionally substituted phenyl is phenyl substituted by a moiety selected from the group consisting of: alkyl, cycloalkyl, aryl, substituted aryl, aralkyl, —(CH₂)ₖOH (where k=1, 2 or 3), —CH₂NH₂, —CH₂NH-alkyl, —CH₂N(alkyl)₂, —CO₂H, —CO₂alkyl, —CONH₂, —CONHalkyl, —CON(alkyl)₂, —OH, alkoxy, aryloxy, —SH, —S-alkyl, —S-aryl, —S(O)alkyl, —S(O)aryl, —SO₂alkyl, —SO₂N(alkyl)₂, —SO₂NHalkyl, —SO₂NH₂, —SO₃H, —SCF₃, —CN, halogen, —CF₃, NO₂, amino, substituted amino, —NHSO₂alkyl, —OCH₂CH₂NH₂, —OCH₂CH₂NHalkyl, —OCH₂CH₂N(alkyl)₂, oxazolidin-2-yl, and alkyl substituted oxazolidin-2-yl.

Even more specific embodiments of Formula 4 include those in which both of R₃₁ and R₃₂ are optionally substituted phenyl, where optionally substituted phenyl is phenyl substituted by a moiety selected from the group consisting of: hydrogen, alkyl, cycloalkyl, aryl, substituted aryl, aralkyl, —(CH₂)ₖOH (where k=1, 2 or 3), —CH₂NH₂, —CH₂NH-alkyl, —CH₂N(alkyl)₂, —CO₂H, —CO₂alkyl, —CONH₂, —CONHalkyl, —CON(alkyl)₂, —OH, alkoxy, aryloxy, —SH, —S-alkyl, —S-aryl, —S(O)alkyl, —S(O)aryl, —SO₂alkyl, —SO₂N(alkyl)₂, —SO₂NHalkyl, —SO₂NH₂, —SO₃H, —SCF₃, —CN, halogen, —CF₃, —NO₂, amino, substituted amino, —NHSO₂alkyl, —OCH₂CH₂NH₂, —OCH₂CH₂NHalkyl, —OCH₂CH₂N(alkyl)₂, oxazolidin-2-yl, and alkyl substituted with oxazolidin-2-yl and R₃₃ is hydrogen, hydroxy, alkoxy, or carboxy are those where R₃₄ is hydroxy or carboxy.

Other more specific embodiments of Formula 4, are those wherein both of R₃₁ and R₃₂ are optionally substituted phenyl where optionally substituted phenyl is phenyl substituted by a moiety selected from the group consisting of: hydrogen, alkyl, cycloalkyl, aryl, substituted aryl, aralkyl, (CH₂)ₖOH (where k=1, 2 or 3), CH₂NH₂, CH₂NH-alkyl, CH₂N(alkyl)₂, CO₂H, CO₂alkyl, CONH₂, CONHalkyl, CON(alkyl)₂, OH, alkoxy, aryloxy, SH, S-alkyl, S-aryl, S(O)alkyl, S(O)aryl, SO₂alkyl, SO₂N(alkyl)₂, SO₂NHalkyl, SO₂NH₂, SO₃H, SCF₃, CN, halogen, CF₃, NO₂, amino, substituted amino, NH₂SO₂alkyl, OCH₂CH₂NH₂, OCH₂CH₂NHalkyl, OCH₂CH₂N(alkyl)₂, oxazolidin-2-yl, and alkyl substituted oxazolidin-2-yl and R₃₃ is hydrogen, hydroxy, alkoxy, or carboxy are those where R₃₄ is hydroxy. These compounds include more specific compounds where the optionally substituted phenyl is phenyl substituted by a moiety selected from the group consisting of: hydrogen, halogen, and alkyl. Still more specific embodiments of the group of compounds just recited are those where the halogen is chloro. Other more specific embodiments are those where the halogen is chloro and the alkyl is methyl.

Among the most useful compounds is (bis(3-chloro-4-methylphenyl)boryloxy)(3-hydroxypyridin-2-yl)methanone, including its pharmaceutically acceptable salts, hydrates, and solvates.

The structures of the invention also permit solvent interactions that may afford structures (such as Formulas 3 and 4) that include atoms derived from the solvent encountered by the compounds of the invention during synthetic procedures and therapeutic uses. Thus, such solvent structures can especially insinuate themselves into at least some of the compounds of the invention, especially between the boron and nitrogen atoms, to increase the ring size of such compounds by one or two atoms. For example, where the boron ring of a structure of the invention comprises 5 atoms, including, for example, the boron, a nitrogen, an oxygen and 2 carbons, insinuation of a solvent atom between the boron and nitrogen would afford a 6- or 7-membered ring. in one example, use of hydroxyl and amino solvents may afford structures containing an oxygen or nitrogen between the ring boron and nitrogen atoms to increase the size of the ring. Such structures are expressly contemplated by the present invention, preferably where R*** is H or alkyl.

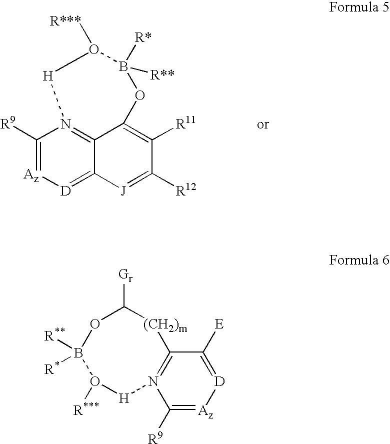

As used herein, the following terms have the stated meaning unless specifically defined otherwise in this application:

By “alkyl” in the present invention is meant straight or branched chain alkyl groups having 1-10 carbon atoms and preferably 1-6 carbon atoms. The terms “lower alkyl”, and “C₁-C₆ alkyl” both refer to alkyl groups of 1-6 carbon atoms. Examples of such alkyl groups include, for instance, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl.

By “substituted alkyl” is meant an alkyl group having from 1 to 5 and preferably 1 to 3 and more preferably 1 substituent selected from alkoxy, substituted alkoxy, aryl, substituted aryl, aryloxy, substituted aryloxy, cycloalkyl, substituted cycloalkyl, heteroaryl, substituted heteroaryl, heterocyclic, substituted heterocyclic, hydroxyl, amino, substituted amino, carboxyl, -carboxyl-alkyl, amido, thiol, alkylthio, substituted alkylthio, arylthio, substituted arylthio, —SO₂-alkyl, —SO₂-amino, —SO₂-substituted amino, —SO₂—OH, —SCF₃, cyano, halo, nitro, and —NHSO₂alkyl.

By “substituted lower alkyl” is meant a lower alkyl group substituted with 1 to 5 and preferably 1 to 3 and more preferably 1 substituent as defined above for substituted alkyl.

By “alkylene” is meant a divalent alkyl group having from 1 to 10 carbon atoms, preferably from 1 to 5 carbon atoms and more preferably 1 to 3 carbon atoms. This term is exemplified by groups such as methylene, 1,2-ethylene, 1,3-n-propylene, 1,4-n-butylene, 2-methyl-1,4-propylene and the like.

By “substituted alkylene” is meant an alkylene group having from 1 to 5 and preferably 1 to 3 and more preferably 1 substituent as defined above for substituted alkyl.

By “alkoxy”, “lower alkoxy”, and “C₁-C₆ alkoxy’ is meant straight or branched chain alkoxy groups having 1-6 carbon atoms, such as, for example, methoxy, ethoxy, propoxy, isopropoxy, n-butoxy, sec-butoxy, tent butoxy, pentoxy, 2-pentyl, isopentoxy, neopentoxy, hexoxy, 2-hexoxy, 3-hexoxy, and 3-methylpentoxy.

By “substituted alkoxy” is meant —O-substituted alkyl.

By “substituted lower alkoxy” is meant a —O-lower alkyl group substituted with 1 to 5 and preferably 1 to 3 and more preferably 1 substituent as defined above for substituted alkyl.

By “alkylcarbonyloxy” is meant —C—(O)-alkyl.

By “hydroxyalkyl” is meant alkyl substituted with hydroxy.

By “hydroxyalkoxy” is meant alkoxy substituted with hydroxy.

By “carboxyalkyloxy” is meant —O-alkyl-COOH and salts thereof.

By “alkyloxycarbonyl” is meant —C(O)—O-alkyl.

By “alkenyl” in the present invention is meant an alkenyl group having from 2 to 6 carbon atoms and more preferably 2 to 4 carbon atoms and having at least 1 and preferably 1 site of alkenyl unsaturation. Examples of alkenyl groups include, for instance, vinyl, allyl, n-but-2-en-1-yl, and the like.

By “substituted alkenyl” is meant an alkenyl group having from 1 to 3 substituents and preferably one substituent selected from alkoxy, substituted alkoxy, cycloalkyl, substituted cycloalkyl, aryl, substituted aryl, aryloxy, substituted aryloxy, heteroaryl, substituted heteroaryl, heterocyclic, substituted heterocyclic, hydroxyl, amino, substituted amino, carboxyl, -carboxyl-alkyl, amido, thiol, alkylthio, substituted alkylthio, arylthio, substituted arylthio, —SO₂-alkyl, —SO₂-amino, —SO₂-substituted amino, —SO₂—OH, —SCF₃, cyano, halo, nitro, —NHSO₂alkyl, and —C(O)SO₂-alkyl with the proviso that any hydroxyl or thiol substitution is not on a vinyl carbon atom.

The terms alkenyl and substituted alkenyl encompass both the cis and trans isomers as well as mixtures thereof.

By “alkynyl” is meant an alkynyl group having from 2 to 6 carbon atoms and more preferably 2 to 4 carbon atoms and having at least 1 and preferably 1 site of alkynyl unsaturation. Examples of alkynyl groups include, for instance, acetylenyl, propargyl, n-but-2-yn-1-yl, and the like.

By “substituted alkynyl” is meant an alkynyl group having from 1 to 3 substituents and preferably one substituent selected from alkoxy, substituted alkoxy, cycloalkyl, substituted cycloalkyl, aryl, substituted aryl, aryloxy, substituted aryloxy, heteroaryl, substituted heteroaryl, heterocyclic, substituted heterocyclic, hydroxyl, amino, substituted amino, carboxyl, -carboxyl-alkyl, amido, thiol, alkylthio, substituted alkylthio, arylthio, substituted arylthio, —SO₂-alkyl, —SO₂-amino, —SO₂-substituted amino, —SO₂—OH, —SCF₃, cyano, halo, nitro, —NHSO₂alkyl, and —C(O)SO₂-alkyl with the proviso that any hydroxyl or thiol substitution is not on an acetylenic carbon atom.

By “amino” is meant —NH₂.

By “substituted amino” is meant as an —NR′R″ group where R′ and R″ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic and substituted heterocyclic or where R′ and R″ and the nitrogen atom bound thereto form a heterocyclic or substituted heterocyclic group with the proviso that R′ and R″ and not both hydrogen.

By “alkylamino” is meant —NH-alkyl.

By “aminoalkyl” is meant -alkylene-NH₂.

By “dialkylamino” is meant —N(alkyl)(alkyl), where each alkyl may be the same or different.

By “(alkylamino)alkyl” is meant -alkylene-NH-alkyl, where each alkyl can be the same or different.

By “(dialkylamino)alkyl” is meant -alkylene-N(alkyl)(alkyl), where each alkyl can be the same or different.

By “arylamino” is meant —NH-aryl, where aryl is defined below.

By “alkylsulfonylamino” is meant —NH—SO₂alkyl.

By “arylsulfonylamino” is meant —NH—SO₂aryl, where aryl is defined below.

By “diarylamino” is meant —N(aryl)(aryl), where each aryl may be the same or different and aryl is defined below.

By “acyloxy” is meant the groups —OC(O)alkyl, —O(C)substituted alkyl, —OC(O)alkenyl, —OC(O)substituted alkenyl, —OC(O)alkynyl, —OC(O)substituted alkynyl, —OC(O)aryl, —OC(O)substituted aryl, —OC(O)cycloalkyl, —O(CO)substituted cycloalkyl, —OC(O)heteroaryl, —OC(O)substituted heteroaryl, —OC(O)heterocyclic, and —OC(O)substituted heterocyclic.

By “alkyloxycarbonyl” is meant —C(O)—Oalkyl.

By “amido” or “carbamoyl” is meant —C(O)amino and —C(O)substituted amino.

By “alkyl carbamoyl” is meant —C(O)—NH-alkyl.

By the term “halogen” or “halo” is meant fluorine, bromine, chlorine, and iodine.

By “cycloalkyl”, e.g., C₃-C₇ cycloalkyl, is meant cycloalkyl groups having 3-7 atoms such as, for example cyclopropyl, cyclobutyl, cyclopentyl, cyclohexyl, and cycloheptyl.

By “substituted cycloalkyl” is meant a cycloalkyl group having from 1 to 3 and preferably one substituent selected from alkyl, substituted alkyl, alkoxy, substituted alkoxy, cycloalkyl, substituted cycloalkyl, aryl, substituted aryl, aryloxy, substituted aryloxy, heteroaryl, substituted heteroaryl, heterocyclic, substituted heterocyclic, hydroxyl, amino, substituted amino, carboxyl, -carboxyl-alkyl, amido, thiol, alkylthio, substituted alkylthio, arylthio, substituted arylthio, —SO₂-alkyl, —SO₂-amino, —SO₂-substituted amino, —SO₂—OH, —SCF₃, cyano, halo, nitro, —NHSO₂alkyl, —C(O)SO₂-alkyl, keto(C═O) and thioketo(C═S).

By the term “cycloalkyloxy” or “substituted cycloalkyloxy” is meant —O-cycloalkyl and —O-substituted cycloalkyl.

By “aryl” is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl, anthryl, or phenanthryl), provided that the point of attachment is to an aromatic carbon atom.

By “substituted aryl” is meant an aryl group having from 1 to 3 and preferably one substituent selected from acyloxy, alkyl, substituted alkyl, alkoxy, substituted alkoxy, cycloalkyl, substituted cycloalkyl, aryl, substituted aryl, aryloxy, substituted aryloxy, heteroaryl, substituted heteroaryl, heterocyclic, substituted heterocyclic, hydroxyl, amino, substituted amino, carboxyl, -carboxyl-alkyl, amido, thiol, alkylthio, substituted alkylthio, arylthio, substituted arylthio, —SO₂-alkyl, —SO₂-amino, —SO₂-substituted amino, —SO₂—OH, —SCF₃, cyano, halo, nitro, —NHSO₂alkyl, and —C(O)SO₂-alkyl. In one embodiment, the substituted aryl group is mono-, di-, or tri-substituted with halo, lower alkyl, lower alkoxy, lower alkylthio, trifluoromethyl, lower acyloxy, aryl, heteroaryl, and hydroxy. Preferred aryl groups include phenyl and naphthyl, each of which is optionally substituted as defined herein.

By “aryloxy” is meant —O-aryl.

By “substituted aryloxy” is meant —O-substituted aryl.

By “arylcarbonyl” is meant —C(O)aryl.

By “aralkyl” is meant the groups -alkylene-aryl, -alkyene substituted aryl, -substituted alkylene-aryl and -substituted alkylene-substituted aryl.

By “carboxyl” or “carboxy” is meant —COOH and salts thereof

“Alkanoyl” or “acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)-cycloalkyl-C(O)—, substituted cycloalkyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

The term “alkanoylamino” refers to the group —NH—C(O)H and —NHC(O)-alkyl, preferably the alkanoyl amino is —NHC(O)-alkyl.

By “heteroaryl” is meant one or more aromatic ring systems of 5-, 6-, or 7-membered rings containing at least one and up to four heteroatoms selected from nitrogen, oxygen, or sulfur. Such heteroaryl groups include, for example, thienyl, furanyl, thiazolyl, imidazolyl, (is)oxazolyl, pyridyl, pyrimidinyl, (iso)quinolinyl, napthyridinyl, benzimidazolyl, and benzoxazolyl. Preferred heteroaryls are thiazolyl, pyrimidinyl, preferably pyrimidin-2-yl, and pyridyl. Other preferred heteroaryl groups include 1-imidazolyl, 2-thienyl, 1-(or 2-)quinolinyl, 1-(or 2-)isoquinolinyl, 1-(or 2-)tetrahydroisoquinolinyl, 2-(or 3-)furanyl and 2-tetrahydrofuranyl.

By “substituted heteroaryl” is meant a heteroaryl group having from 1 to 3 and preferably one substituted as defined above for substituted aryl.

By “heteroaryloxy” and “substituted heteroaryloxy” is meant —O-heteroaryl and —O-substituted heteroaryl, respectively.

By “heteroaralkyl” is meant the groups -alkylene-heteroaryl, -alkylene substituted heteroaryl, -substituted alkylene-heteroaryl and -substituted alkylene-substituted heteroaryl.

By “heterocyclic” or “heterocycle” or “heterocyclyl” or “heterocycloalkyl” or “cycloheteroalkyl” is meant refers to a saturated or unsaturated group having a single ring or multiple condensed rings, from 1 to 10 carbon atoms and from 1 to 4 hetero atoms selected from the group consisting of nitrogen, sulfur or oxygen within the ring wherein, in fused ring systems, one or more the rings can be aryl or heteroaryl provided that the point of attachment is to a heterocyclic ring atom.

By “substituted heterocyclic” is meant a heterocycle group that is substituted with from 1 to 3 and preferably 1 substituent of the same substituents as defined for substituted cycloalkyl.

By “heterocyclyloxy” is meant —O-heterocyclyl.

By “thiol” or “thio” is meant —SH.

By “alkylthio” is meant —S-alkyl.

By “substituted alkylthio” is meant —S-substituted alkyl.

By “arylthio” is meant —S-aryl.

By “substituted arylthio” is meant —S-substituted aryl.

By “heteroarylthio” is meant —S-heteroaryl.

By “cyano” is meand —CN.

By “formyl” is meant —C(═O)H or —CHO.

By “thioformyl” is meant —C(═S)H or —CHS.

By “sulfonyl” is meant —SO₃H.

By “alkylsulfonyl” is meant —SO₂alkyl.

By “arylsulfonyl” is meant —SO₂aryl.

By “heteroarylsulfonyl” is meant —SO₂heteroaryl.

By “alkylsulfinyl” is meant —SOalkyl.

By “arylsulfinyl” is meant —SOaryl.

By “heteroarylsulfinyl” is meant —SOheteroaryl.

By “sulfamoyl” is meant —SO₂—NH₂.

By “sulfamoyloxy” is meant —O—SO₂—NH₂.

By “alkylsulfamoyl” is meant —SO₂—NH-alkyl.

By “dialkylsulfamoyl” is meant —SO₂—N(alkyl)(alkyl), where each alkyl can be the same or different.

By “thiocarboxyl” is meant —C(═S)OH, —C(═O)SH, or —C(═S)SH.

By “thiocarbamoyl” is meant —C(═S)amino and —C(═S)substituted amino.

The term “aromatic ring” refers to optionally substituted aryl groups and optionally substituted heteroaryl groups.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that, for example, substituted aryl groups are limted to -substituted aryl-(substituted aryl)-substituted aryl. Impermissible substitutions are not contemplated by the invention.

By “ligand” is meant a nitrogen-containing aromatic system that is capable of forming a dative bond with the Lewis acidic boron center, while appended as a borinate ester moiety. Such ligands are known to those trained in the arts. Examples are shown in the structures below:

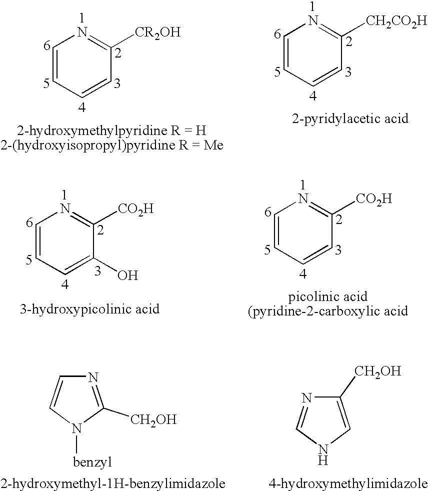

For oral administration, the compounds can be formulated readily by combining the active compounds with pharmaceutically acceptable carriers well known in the art. Such carriers enable the compounds of the invention to be formulated as tablets, pills, capsules, liquids, gels, syrups, slurries, suspensions and the like, for oral ingestion by a patient to be treated. Pharmaceutical preparations for oral use can be obtained with solid excipient, optionally grinding a resulting mixture, and processing the mixture of granules, after adding suitable auxiliaries, if desired, to obtain tablets. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration. For buccal administration, the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin, for use in an inhaler can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions. Alternatively, the active ingredient can be in powder form for constitution with a suitable vehicle, e.g., sterile pyrogen-free water, before use. The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The cosolvent system can be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of polysorbate 80; the fraction size of polyethylene glycol can be varied; other biocompatible polymers can replace polyethylene glycol, e.g., polyvinyl pyrrolidone; and other sugars or polysaccharides can substitute for dextrose.

Alternatively, other delivery systems for hydrophobic pharmaceutical compounds can be employed. Liposomes and emulsions are well known examples of delivery vehicles or carriers for hydrophobic drugs. Certain organic solvents such as dimethyl sulfoxide also can be employed, although usually at the cost of greater toxicity. Additionally, the compounds can be delivered using a sustained-release system, such as semipermeable matrices of solid hydrophobic polymers containing the therapeutic agent. Various sustained-release materials have been established and are well known by those skilled in the art. Sustained release capsules can, depending on their chemical nature, release the compounds for a few weeks up to over 100 days. Depending on the chemical nature and the biological stability of the therapeutic reagent, additional strategies for protein and nucleic acid stabilization can be employed.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The compounds can be provided as salts with pharmaceutically compatible counterions. Pharmaceutically compatible salts can be formed with many acids, including but not limited to hydrochloric, sulfuric, acetic, lactic, tartaric, malic, succinic, phosphoric, hydrobromic, sulfinic, formic, toluenesulfonic, methanesulfonic, nitric, benzoic, citric, tartaric, maleic, hydroiodic, alkanoic such as acetic, HOOC—(CH₂)ᵣ—CH₃ where r is 0-4, and the like. Salts tend to be more soluble in aqueous or other protonic solvents that are the corresponding free base forms. Non-toxic pharmaceutical base addition salts include salts of bases such as sodium, potassium, calcium, ammonium, and the like. Those skilled in the art will recognize a wide variety of non-toxic pharmaceutically acceptable addition salts.

Pharmaceutical compositions of the compounds can be formulated and administered through a variety of means, including systemic, localized, or topical administration.

For topical administration, the compounds can be formulated readily by combining the active compounds with pharmaceutically acceptable carriers well known in the art. Such carriers enable the compounds of the invention to be formulated as gels, slurries, suspensions, creams, and ointments for topical applications. if desired, disintegrating agents can be added, such as the cross-linked polyvinyl pyrrolidone, agar, or alginic acid or a salt thereof such as sodium alginate.

Techniques for formulation and administration can be found in Remington's Pharmaceutical Sciences, Mack Publishing Co., Easton, Pa., The mode of administration can be selected to maximize delivery to a desired target site in the by. Suitable routes of administration can, for example, include oral, rectal, transmucosal, transcutaneous, or intestinal administration. Parenteral delivery, including intramuscular, subcutaneous, and intramedullary injections, as well as intrathecal, direct intraventricular, intravenous, intraperitoneal, intranasal, or intraocular injections are also contemplated.

Alternatively, one can administer the compound in a local rather than systemic manner, for example, by injection of the compound directly into a specific tissue, often in a depot or sustained release formulation.

Pharmaceutical compositions suitable for use include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For any compound used in the method of the invention, the therapeutically effective dose can be estimated initially from cell culture assays, as disclosed herein. For example, a dose can be formulated in animal models to achieve a circulating concentration range that includes the ED₅₀ (effective dose for 50% increase) as determined in cell culture, i.e., the concentration of the test compound which achieves a half maximal inhibition of bacterial cell growth. Such information can be used to more accurately determine useful doses in humans.

It will be understood, however, that the specific dose level for any particular patient will depend upon a variety of factors including the activity of the specific compound employed, the age, body weight, general health, sex, diet, time of administration, route of administration, and rate of excretion, drug combination, the severity of the particular disease undergoing therapy and the judgment of the prescribing physician.

For administration to non-human animals, the drug or a pharmaceutical composition containing the drug may also be added to the animal feed or drinking water. It will be convenient to formulate animal feed and drinking water products with a predetermined dose of the drug so that the animal takes in an appropriate quantity of the drug along with its diet. It will also be convenient to add a premix containing the drug to the feed or drinking water approximately immediately prior to consumption by the animal.

Preferred compounds for the invention anti-parasitic use will have certain pharmacological properties. Such properties include, but are not limited to, oral bioavailability, low toxicity, low serum protein binding and desirable in vitro and in vivo hall-lives. Assays may be used to predict these desirable pharmacological properties. Assays used to predict bioavailability include transport across human intestinal cell monolayers, including Caw-2 cell monolayers. Serum protein binding may be predicted from albumin binding assays. Such assays are described in a review by Oravcová, et al. (1996, J. Chromat. B 677: 1-27). Compound half-life is inversely proportional to the frequency of dosage of a compound. In vitro half-lives of compounds may be predicted from assays of microsomal half-life as described by Kuhnz and Gieschen (Drug Metabolism and Disposition, (1998) volume 26, pages 1120-1127).

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds that exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of the patient's condition. (See, e.g., Fingl, et al., 1975, in The Pharmacological Basis of Therapeutics, Ch. 1, p. 1).

Dosage amount and interval can be adjusted individually to provide plasma levels of the active moiety that are sufficient to maintain bacterial cell growth inhibitory effects. Usual patient dosages for systemic administration range from 100-2000 mg/day. Stated in terms of patient body surface areas, usual dosages range from 50-910 mg/m²/day. Usual average plasma levels should be maintained within 0.1-1000 M. In cases of local administration or selective uptake, the effective local concentration of the compound cannot be related to plasma concentration.

This invention relates to composition and methods for the treatment of diseases of both animals and human caused by pathogenic parasites. The anti-parasitic compounds of the invention are useful for the treatment of diseases of both animals and humans, including but not limited to malaria, Chagas' disease, Leishmaniasis, African sleeping sickness (African human trypanosomiasis), giardiasis, toxoplasmosis, amebiasis and cryptosporidiosis.

The disclosures in this application of all articles and references, including patents and patent applications, are incorporated herein by reference in their entirety.

The compounds of this invention comprise a novel class of anti-parasitic agents. Medically-important parasitic species that are susceptible to these agents include, but not limited to Plasmodium falciparum, P. vivax, P. ovale P. malariae, P. berghei, Leishmania donovani, L. infantum, L. chagasi, L. mexicana, L. amazonensis, L. venezuelensis, L. tropics, L. major, L. minor, L. aethiopica, L. Biana braziliensis, L. (V.) guyanensis, L. (V.) panamensis, L. (V.) peruviana, Trypanosoma brucei rhodesiense, T brucei gambiense, T. cruzi, Giardia intestinalis, G. lambda, Toxoplasma gondii, Entamoeba histolytica, Trichomonas vaginalis, Pneumocystis carinii, and Cryptosporidium parvum.

### 5 EXAMPLES

### 5.1 General Considerations

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those skillful in the art will have sufficient knowledge of such systems and methodologies so as to be able, without undue experimentation, to make such substitutions as will optimally serve their purposes in using the methods and procedures disclosed herein.

The invention is described in more detail in the following non-limiting examples. It is to be understood that these methods and examples in no way limit the invention to the embodiments described herein and that other embodiments and uses will no doubt suggest themselves to those skilled in the art.

The compounds are evaluated for their anti-parasitic activity against Plasmodium falciparum, Leishmania donovani, Trypanosome brucei rhodesiense, and T. cruzi in the in vitro screening models for malaria, sleeping sickness, leishmaniasis (both axenic and in macrophage) and Chaga's disease. The protocols for these models are described below.

### 5.2 Protocols For Anti-Parasitic Activity In Vitro: Malaria: In Vitro Screening Model

### 5.2.1 Parasite Cultures

Two strains of P. falciparum K1 is used in this study. K1 (a clone originating from Thailand) is resistant to chloroquine and pyrimethamine. The strains are maintained in RPMI 1640 medium with 0.36 mM hypoxanthine, supplemented with 25 mM N 2-hydroxyethyipiperazine-N-2-ethane-sulphonic acid (HEPES), 25 mM NaHCO₃, neomycin (100 U/mL) and 5 g/L of ALBUMAX® II (lipid-rich bovine serum albumin, GIBCO, Grand Island, N.Y., USA), together with 5% washed human A+ erythrocytes. All cultures and assays are conducted at 37° C. under an atmosphere of 4% CO₂, 3% O₂ and 93% N₂. Cultures are kept in incubation chambers filled with the gas mixture. Subcultures are diluted to a parasitemia of between 0.1 and 0.5% and the medium is changed daily.

### 5.2.2 Drug Sensitivity Assays

Antimalarial activity is assessed using an adaptation of the procedures described by Desjardins et al. (Antimicrob. Agents Chemother. 16:710-718, 1979), and Matile and Pink (In: Lefkovits, I. and Pernis, B. (Eds.), Immunological Methods Vol. IV, Academic Press, San Diego, pp. 221-234, 1990).

Stock drug solutions are prepared in 100% dimethylsulfoxide (DMSO) at 10 mg/mL, and heated or sonicated if necessary to dissolve the sample. After use, the stocks are kept at −20° C. For the assays, the compound is further diluted in serum-free culture medium and finally to the appropriate concentration in complete medium without hypoxanthine. The DMSO concentration in the wells with the highest drug concentration does not exceed 1%.

Assays are performed in sterile 96-well microtiter plates, each well containing 200 μl of parasite culture (0.15% parasitemia, 2.5% hematocrit) with or without serial drug solutions. Seven 2-fold dilutions are used, covering a range from 5 μl/mL to 0.078 μg/mL. For active compounds the highest concentration is lowered (e.g., to 100 μg/mL); for plant extracts the highest concentration is increased to 50 μg/mL. Each drug is tested in duplicate and the assay is repeated for active compounds showing an IC₅₀ below 1.0 μg/mL. After 48 hours of incubation at 37° C., 0.5 μCi ³H-hypoxanthine is added to each well. Cultures are incubated for a further 24 h before being harvested onto glass-fiber filters and washed with distilled water. The radioactivity is counted using a Betaplate liquid scintillation counter (Wallac, Zurich, Switzerland). The results are recorded as counts per minute per well at each drug concentration and expressed as percentage of the untreated controls. IC₅₀ values are calculated from the sigmoidal inhibition curves using Microsoft EXCEL.

### 5.3 African Sleeping Sickness: In Vitro Screening Model

### 5.3.1 Parasite Cultures

Three strains of Trypanosoma brucei spp. may be used in this study: (a) T. b. rhodesiense STIB 900 (a clone of a population isolated in 1982 from a patient in Tanzania), which is known to be susceptible to all currently used drugs; (b) T. b. gambiense STIB 930 (a derivative of strain TH1178E (031), isolated in 1978 from a patient in Ivory Coast), which is known to be sensitive to all drugs used; and (c) T. b. brucei STIB 950 (a clone of a population isolated in 1985 from a bovine in Somalia), which shows drug resistance to diminazene, isometamidium and quinapyramine.

Bloodstream from trypomastigotes of the strains (a) and (c) are maintained in Minimal Essential Medium (MEM) with Earle's salts supplemented according to Baltz, et al., (EMBO J. 4:1273-1277, 1985) with 25 mM N-2-hydroxyethylpiperazine-N′-2-ethane-sulphonic acid (HEPES), 1 g/L additional glucose, 1% MEM non-essential amino acids (100×), 0.2 mM 2-mercaptoethanol, 2 mM sodium pyruvate, 0.1 mM hypoxanthine and 15% heat-inactivated horse serum.

Bloodstream from trypomastigotes of strain (b) are maintained in MEM with Earle's salts supplemented with 25 mM HEPES, 1 g/L additional glucose, 1% MEM non-essential amino acids (100×), 0.2 mM 2-mercaptoethanol, 2 mM sodium pyruvate, 0.1 mM hypoxanthine, 0.05 mM bathocuproine disulphonic acid, 0.15 mM L-cysteine and 15% heat-inactivated pooled human serum.

All cultures and assays are conducted at 37° C. under an atmosphere of 5% CO₂ in air.

### 5.3.2 Drug Sensitivity Assays

Stock drug solutions are prepared in 100% dimethylsulfoxide (DMSO) (unless otherwise suggested by the supplier) at 10 mg/mL, and heated or sonicated if necessary to dissolve the sample. After use the stocks are kept at −20° C. For the assays, the compound is further diluted in serum-free culture medium and finally to the appropriate concentration in complete medium without hypoxanthine. The DMSO concentration in the wells with the highest drug concentration does not exceed 1%.

Assays are performed in 96-well microtiter plates, each well containing 100 μL of culture medium with 8×10³ bloodstream forms with or without a serial drug dilution. The highest concentration for the test compounds is 90 μg/mL. Seven 3-fold drug dilutions are used, covering a range from 90 μg/mL to 0.123 μg/mL. Each drug is tested in duplicate. Active compounds are tested twice for confirmation. The final result is the mean of the four individual IC₅₀ values. After 72 hrs of incubation, the plates are inspected under an inverted microscope to assure growth of the controls and sterile conditions.

Ten μL of Alamar Blue (12.5 mg resazurin dissolved in 100 mL distilled water) are now added to each well and the plates are incubated for another 2 hours. Then the plates are read with a Spectramax GEMINI XS microplate fluorometer (Molecular Devices Cooperation, Sunnyvale, Calif., USA) using an excitation wavelength of 536 nm and an emission wave length of 588 nm.

IC₅₀ values are determined using the microplate reader software Softmax Pro (Molecular Devices Cooperation, Sunnyvale, Calif., USA).

### 5.4 Chagas Disease: In Vitro Screening Model Parasite and Cell Cultures

Trypanosoma cruzi Tulahuen C2C4 strain, containing the β-galactosidase (Lac Z) gene (Buckner et al., Antimicrob. Agents Chemother 40:2592-2597, 1996) is used for this study.

The infective amastigote and trypomastigote stages are cultivated in L-6 cells (a rat skeletal myoblast cell line) in RPMI 1640 medium supplemented with 2 mM L-glutamine and 10% heat-inactivated fetal bovine serum in 12.5 cm² tissue culture flasks. Amastigotes develop intracellularly, differentiate into trypomastigotes, and leave the host cell. These trypomastigotes infect new L-6 cells and are the stages used to initiate an infection in the assay. All cultures and assays are conducted at 37° C. under an atmosphere of 5% CO₂ in air.

### 5.4.1 Drug Sensitivity Assays

Stock drug solutions are prepared in 100% dimethylsulfoxide (DMSO) (unless otherwise suggested by the supplier) at 10 mg/mL, and heated or sonicated if necessary to dissolve the sample. After use the stocks are kept at −20° C. For the assays, the compound is further diluted to the appropriate concentration using complete medium. The DMSO concentration in the wells with the highest drug concentration does not exceed 1%.

Assays are performed in sterile 96-well microtiter plates, each well containing 100 μl medium with 2×10³ L-6 cells. After 24 hours, 50 mL of a trypanosome suspension containing 5×10³ trypomastigote bloodstream forms from culture are added to the wells. Forty-eight hours later, the medium is removed from the wells and replaced by 100 L fresh medium, with or without a serial drug dilution. At this point the L-6 cells should be infected with amastigotes and no free trypomastigotes should be in the medium. Seven 3-fold drug dilutions are used, covering a range from 90 μg/mL to 0.123 μg/mL. Each drug is tested in duplicate. Active compounds are tested twice for confirmation. After 96 hours of incubation the plates are inspected under an inverted microscope to assure growth of the controls and sterility.

Then the substrate CPRG/Nonidet (50 μl) is added to all wells. A colour reaction will become visible within 2-6 hours and can be read photometrically at 540 nm. Data are transferred into a graphics program (e.g., EXCEL), sigmoidal inhibition curves determined and IC₅₀ values calculated.

### 5.5 Leishmaniasis: Axenic In Vitro Screening Model

### 5.5.1 Parasite and Cell Cultures

The Leishmania donovani strain MHOM/ET1671L82 (obtained from Dr. S. Croft, London School of Hygiene and Tropical Medicine) is used. The strain is maintained in the Syrian Golden hamster. Amastigotes are collected from the spleen of an infected hamster. Amastigotes are grown in axenic culture at 37° C. in SM medium (Cunningham I., J. Protozoal. 24:325-329, 1977) at pH 5.4 supplemented with 10% heat-inactivated fetal bovine serum (FBS) under an atmosphere of 5% CO₂ in air.

### 5.5.2 Drug Sensitivity Assays

Stock drug solutions are prepared in 100% dimethylsulfoxide (DMSO) (unless otherwise suggested by the supplier) at 10 mg/mL, and heated or sonicated if necessary to dissolve the sample. After use the stocks are kept at −20° C. For the assays, the compound is further diluted to the appropriate concentration using complete medium. The DMSO concentration in the wells with the highest drug concentration does not exceed 1%.

Assays are performed in 96-well flat-bottom microtiter plates (Costar, Corning Inc.), each well containing 100 μl of culture medium with 105 amastigotes from axenic culture with or without a serial drug dilution. Concentration of amastigotes is determined in a CASY cell analyzing system (Scharfe System, Reutlingen, Germany). Before the amastigotes are counted, the parasite culture is passed twice through a 22-gauge needle to break up clusters of amastigotes.

The highest concentration for the test compounds is 90 μg/mL. Seven 3-fold dilutions are used, covering a range from 30 μg/mL to 0.041 μg/mL. Each drug is tested in duplicate. Active compounds are tested twice for confirmation. After 72 hours of incubation, the plates are inspected under an inverted microscope to assure growth of the controls and sterile conditions.

Ten μL of Alamar Blue (12.5 mg resazurin dissolved in 100 mL distilled water) are then added to each well and the plates are incubated for another 2 hours. Then the plates are read with a Spectramax GEMINI XS microplate fluorometer (Molecular Devices Cooperation, Sunnyvale, Calif., USA) using an excitation wave length of 536 nm and an emission wave length of 588 nm.

Data are analyzed using the microplate reader software Softmax Pro (Molecular Devices Cooperation, Sunnyvale, Calif., USA). Decrease of fluorescence (i.e. inhibition) is expressed as percentage of the fluorescence of control cultures and plotted against the drug concentrations. The IC50 value is calculated from the sigmoidal inhibition curve by the software program.

### 5.6 Leishmaniasis: Macrophage In Vitro Screening Model

### 5.6.1 Parasite and Cell Cultures

The Leishmania donovani strain MHOM/ET/67/L82 (obtained from Dr. S. Croft, London School of Hygiene and Tropical Medicine) is used. The strain is maintained in the Syrian Golden hamster. Amastigotes are collected from the spleen of an infected hamster. Amastigotes are grown in axenic culture at 37° C. in SM medium (Cunningham, L., J. Protozool. 24:325-329, 1977) at pH 5.4 supplemented with 10% heat-inactivated fetal bovine serum (FBS) under an atmosphere of 5% CO₂ in air.

Primary peritoneal macrophages from NMRI mice are collected one day after stimulation of macrophage production with an intraperitoneal injection of 2 mL of a 2% potato starch suspension (FLUKA, Switzerland). All cultures and assays are done at 37° C. under an atmosphere of 5% CO₂ in air.

### 5.6.2 Drug Sensitivity Assays

Stock drug solutions are prepared in 100% dimethylsulfoxide (DMSO) (unless otherwise suggested by the supplier) at 10 mg/mL, and heated or sonicated if necessary to dissolve the sample. After use the stocks are kept at −20° C. For the assays, the compound is further diluted to the appropriate concentration using complete medium. The DMSO concentration in the wells with the highest drug concentration does not exceed 1%.

Assays are performed in sterile 16-well chamber slides (LabTek, Nalgene/Nunc Int.) To each well are added 100 μL of a murine macrophage suspension (4×10⁵/mL) in RPMI 1640 medium containing bicarbonate and N-2-hydroxyethylpiperazine-N′-2-ethanesulphonic acid (HEPES) and supplemented with 10% heat inactivated FBS (RPMI/FBS). After 24 hrs, 100 μL of a suspension containing amastigotes (1.2×10⁶/mL) are added to each well, giving a 3:1 ratio of amastigotes to macrophages. The amastigotes are harvested from an axenic amastigote culture and suspended in RPMI/FBS. 24 hrs later, the medium containing free amastigotes is removed, the cells are washed once with medium, and fresh medium containing drug dilutions (four 3-fold dilutions for each compound) is added. In this way, four compounds can be tested on one 16-well tissue culture slide. Untreated wells serve as controls. Parasite growth in the presence of the drug is compared to control wells. After 4 days of incubation, the culture medium is removed and the slides are fixed with methanol for 10 min and then stained with a 10% Giemsa solution. Infected and non-infected macrophages are counted in the control cultures and those exposed to the serial drug dilutions. The infection rates are determined. The results are expressed as percent reduction in parasite burden compared to control wells, and the IC₅₀ is calculated by linear regression analysis (EXCEL Microsoft).

### 5.7 In Vivo Efficacy Mouse Malaria Model [P. Berghei (Ankh Strain)]

Test compounds will be compared to chloroquine, artemisinin, mefloquine as follows: NMRI mice, SPF, females, 2 g

### 5.7.1 Day 0

From a donor mouse with approximately 30% parasitemia, heparinized blood is taken and diluted in physiological saline to 10⁸ parasitized erythrocytes per mL. An aliquot of 0.2 mL (=2×10⁷ parasitized erythrocytes) of this suspension is injected intravenously (i.v.) into experimental groups of 4 mice.

Two-to-four hours post-infection, the experimental groups are treated with a single dose by the subcutaneous or the oral route. The compounds are prepared at an appropriate concentration, as a solution or suspension containing 3% ethanol and 7% Tween 80 or in SSV (standard suspending vehicle):

| | |
|---|---|
| Na-CMC (carboxymethylcellulose) | 5 g |
| Benzyl alcohol | 5.0 mL |
| Tween 80 | 4.0 mL |
| 0.9% aqueous NaCl solution | 1.0 L |

### 5.7.2 Days 1 to 3

At 24-, 48- and 72 hours post-infection the experimental groups of mice are treated again with the same dose and by the same route as on Day 0.

### 5.7.3 Day 4

Twenty-four hours after the last treatment (72 hours post-infection) blood smears from all animals are prepared and stained with Giemsa. Parasitemia is determined microscopically by counting 400 red blood cells (“rbcs”), for low parasitemias (<1%) up to 4,000 rbc's have to be counted. The difference between the mean value of the control group (taken as 100%) and those of the experimental groups is calculated and expressed as % Reduction where:

$$\% Reduction = 100 - [ \frac{( mean treated )}{( mean control )} \times 100 ]$$

Additional smears will be taken on day 5 and day 6; and parasitemia will be determined and the activity calculated. Survival time (in days) is recorded, and the mean survival time is calculated. Adverse effects are recorded. Four mice/compound and a minimum of 12 mice/study (vehicle and positive controls run concurrently) are used.

### 5.8 Borinate Complexes

The above procedures were used to obtain the results in the following tables. Representative anti-parasitic data for the compounds 10 to 49 is shown in Table 1 as IC₅₀ (50% Inhibitory Concentration) with the values expressed as micrograms per mL. The in vivo animal efficacy data for a few selected compounds is given in Table 2. A preferred embodiment of the methods of the invention utilizes any one or more of the compounds in Tables 1 and 2. Thus, the invention provides anti-parasitic compounds that are generically called borinic acid complexes or esters, most preferably derived from disubstituted borinic acids.

The synthesis of the compounds of the invention is accomplished in several formats. Reaction scheme #1 demonstrate the synthesis of the intermediate borinic acids, and their subsequent conversion to the desired borinic acid complexes. When R* and R** are identical, the reaction of two equivalents of an aryl magnesium halide (or aryl lithium) with trialkyl borate, followed by acidic hydrolysis affords the desired borinic acid 5. When R* and R** are not identical, the reaction of an equivalent of an aryl magnesium halide (or aryl lithium) with appropriate aryl(dialkoxy)borane (4), heteroaryl(dialkoxy)borane or alkyl(dialkoxy)borane (alkoxy group comprised of methoxy, ethoxy, isopropoxy, or propoxy moiety), followed by acidic hydrolysis affords the unsymmetrical borinic acids 6 in excellent yields. Where applicable, the reaction of the alkylene esters (3, T=single bond, CH₂, CMe₂) with the appropriate organocerium, organolithium, organomagnesium or equivalent reactant is convenient.

As shown in Scheme 1, the borinic acid complexes are obtained from the precursor borinic acids by reaction with one equivalent of the desired heterocyclic ligand in suitable solvents (i.e., ethanol, isopropanol, dioxane, ether, toluene, dimethylformamide, N-methylpyrrolidone, or tetrahydrofuran).

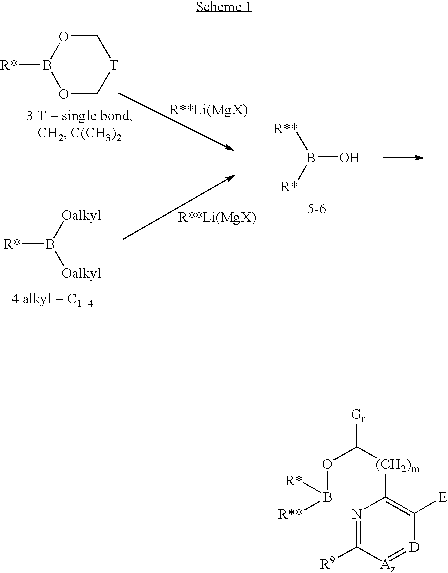

In certain situations, compounds of the invention may contain one or more asymmetric carbon atoms, so that the compounds can exist in different stereoisomeric forms. These compounds can be, for example, racemates or optically active forms. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral HPLC column.

Representative compounds of the present invention include, but are not limited to the compounds disclosed herein and their pharmaceutically acceptable acid and base addition salts. In addition, if the compound of the invention is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. In a preferred embodiment, the compounds of the invention comprise any of compounds 10-144, especially compounds 10-49 (Table 1), and variants thereof. The columns “R*”, “R**”, and “LIGAND” are defined with respect to the structure below. The abbreviation “DMISO” stands for 4,4-dimethyl-4,5-dihydrooxazol-2-yl.

TABLE 1

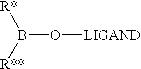

Antiparasitic Profile Against Select Parasites

| | | | | P. | T. b. | | |
|---|---|---|---|---|---|---|---|
| CMPD | R* | R** | LIGAND | falciparum | rhodesiense | T. cruzi | L. donovani |
| | | | | IC₅₀ | IC₅₀ | IC₅₀ | IC₅₀ |
| | | | | mcg/ml | mcg/ml | mcg/ml | mcg/ml |
| 10 | 4-Me-3-Cl-Ph | Ph | quinolin-8-yl | 1.5 | 0.52 | 4.2 | 0.38 |
| 14 | 4-Cl-Ph | 3-F-Ph | quinolin-8-yl | 1.9 | 0.5 | 5.7 | 0.37 |
| 12 | 3-Cl-4-F-Ph | 3-Cl-4-F-Ph | quinolin-8-yl | 1.5 | 0.52 | 5.6 | 0.68 |
| 13 | 3-DMISO-Ph | 3-DMISO-Ph | quinolin-8-yl | 2 | 0.5 | 8.7 | 0.82 |
| 14 | 3-Cl-Ph | 4-F-Ph | quinolin-8-yl | 1.93 | 0.28 | 0.83 | 0.48 |
| 15 | 4-Cl-Ph | 4-F-Ph | quinolin-8-yl | 1.82 | 0.28 | 0.906 | 0.5 |
| 18 | 3-Cl-Ph | 3-Cl-Ph | 5-cyano-quinolin- | 5 | 1.7 | 21 | 0.19 |
| | | | 8-yl | | | | |
| 17 | 3-Cl-Ph | 3-F-Ph | quinolin-8-yl | 1.77 | 0.18 | 1.065 | 0.42 |
| 18 | 3-Cl-Ph | 3-DMISO-Ph | quinolin-8-yl | 1.81 | 0, 43 | 1.65 | 0.50 |
| 19 | 3-F-Ph | 3-DMISO-Ph | quinolin-8-yl | 1.89 | 0.32 | 1.335 | 0.66 |
| 20 | 3-DMISO-Ph | cyclopropyl | quinolin-8-yl | 1.84 | 0.245 | 0.86 | 0.55 |
| 21 | 4-DMISO-Ph | vinyl | quinolin-8-yl | 1.77 | 0.215 | 1.45 | 0.52 |
| 22 | 4-F-Ph | 4-NC-Ph | quinolin-8-yl | 1.46 | 0.325 | 0.845 | 0.34 |
| 23 | 4-(4,5- | vinyl | quinolin-8-yl | 1.77 | 0.299 | 1.82 | 0.55 |
| | dihydrooxazol- | | | | | | |
| | 2-yl)Ph | | | | | | |
| 24 | 3-NC-4-F-Ph | vinyl | quinolin-8-yl | 1.73 | 0.258 | 1.58 | 0.19 |
| 25 | 3-ClPh | 2-Me-Ph | quinolin-8-yl | 1.81 | 0.295 | 0.905 | 0.41 |
| 26 | 3-Ph | 4-NC-Ph | quinolin-8-yl | 1.40 | 0.29 | 0.785 | 0.41 |
| 27 | 3-Cl-Ph | 3-MeO-4- | quinolin-8-yl | 1.7 | 0.52 | 3.2 | 0.47 |
| | | MeO-Ph | | | | | |
| 26 | 3-F-Ph | 4-NC-Ph | quinolin-8-yl | 1.44 | 0.21 | 12 | 0.55 |
| 29 | 3-F-Ph | 3-NC-Ph | quinolin-8-yl | 1.83 | 0.33 | 0.68 | 0.4 |
| 30 | 3-F-Ph | 2-Cl-Ph | quinolin-8-yl | 1.74 | 0.305 | 0.82 | 0.58 |
| 31 | 3-Me-4-Cl-Ph | 3-NC-Ph | quinolin-8-yl | 1.83 | 0.28 | 0.79 | 0.83 |
| 32 | 2,5-di-F-Ph | 3-NC-Ph | quinolin-8-yl | 1.82 | 0.41 | 0.73 | 0.41 |
| 33 | 2-Ph | 3-NC-Ph | quinolin-8-yl | 1.79 | 0.3 | 1.39 | 0.61 |
| 34 | 3-Me-4-Cl-Ph | 4-NC-Ph | quinolin-8-yl | 1.39 | 0.23 | 0.82 | 0.07 |
| 35 | 2,5-di-F-Ph | 4-NC-Ph | quinolin-8-yl | 1.74 | 0.275 | 0.705 | 0.38 |
| 36 | 2-ClPh | vinyl | quinolin-8-yl | 1.5 | 0.18 | 0.785 | 0.39 |
| 37 | 3-NC-Ph | vinyl | quinolin-8-yl | 1.44 | 0.225 | 1.125 | 0.25 |
| 38 | 4-NC-Ph | vinyl | quinolin-8-yl | 0.9 | 0.2305 | 1.51 | 0.47 |
| 39 | 3-F-Ph | vinyl | quinolin-8-yl | 122 | 0.201 | 1.41 | 0.34 |
| 40 | 4-Cl-Ph | 2-F-5-F-Ph | quinolin-8-yl | 1.72 | 0.295 | 0.89 | 0.59 |
| 41 | 4-Cl-Ph | 4-MeO-3- | quinolin-8-yl | 1.63 | 0.38 | 0.935 | 0.29 |
| | | F-Ph | | | | | |
| 42 | 4-Cl-Ph | 2-F-3-F-Ph | quinolin-8-yl | 1.83 | 0.47 | 1.045 | 0.43 |
| 43 | 2-F-4-Cl-Ph | 3-F-Ph | quinolin-8-yl | 1.83 | 0.4 | 0.825 | 0.02 |
| 44 | 4-Cl-Ph | 3,5-di-F-Ph | quinolin-8-yl | 1.87 | 0.275 | 0.71 | 0.5 |
| 45 | 3-MeO-4-Cl-Ph | 3-F-Ph | quinolin-8-yl | 1.84 | 0.48 | 1.145 | 0.50 |
| 46 | 3-Cl-4-Cl-Ph | 3-F-Ph | quinolin-8-yl | 1, 81 | 0.51 | 0.84 | 0.44 |
| 47 | 4-Cl-Ph | 3-F-5-CF₃-Ph | quinolin-8-yl | 2.07 | 0.5 | 1.7 | 0.51 |
| 48 | 3,5-di-F-Ph | vinyl | quinolin-8-yl | 1.34 | 0.218 | 2.23 | 0.21 |
| 49 | 3-pyridyl | vinyl | quinolin-8-yl | 1.58 | 0.2185 | 1.345 | 0.45 |
| 50 | 4-Cl-Ph | 4-Cl-Ph | 6-aminopyridin-2- | 0.6115 | 9.45 | 0.135 | 0.23 |
| | | | carbonyl | | | | |
| 60 | 3-Cl-Ph | 3-Cl-Ph | 6-aminopyridin-2- | 2.139 | 6.95 | 2.79 | 0.76 |
| | | | carbonyl | | | | |
| 61 | 4-Cl-3-Me-Ph | 4-Cl-3-Me-Ph | pyridin-2-carbonyl | 2.873 | 12.5 | 1.535 | 0.39 |
| 62 | 3-Cl-Ph | 3-Cl-Ph | 6-AcNHpyridin-2- | 3.774 | 16.5 | 5.1 | 0.64 |
| | | | carbonyl | | | | |
| 63 | 3-Cl-Ph | 3-Cl-Ph | 6-NH₂-3-OH- | 3.544 | 14.55 | 5.65 | 0.58 |
| | | | pyridin-2-carbonyl | | | | |
| 64 | 4-Cl-Ph | 4-Cl-Ph | 6-NH₂-3-OH- | 3.063 | 8.5 | 4.25 | 1.1 |
| | | | pyridin-2-carbonyl | | | | |
| 65 | 3-Cl-4-Me-Ph | 3-Cl-4-Me-Ph | 5-Bu-pyridin-2- | 1.916 | 19.6 | 1.85 | 0.49 |
| | | | carbonyl | | | | |
| 66 | 3-Cl-4-Me-Ph | 3-Cl-4-Me-Ph | 5-CO₂H-pyridin-2- | 3.61 | 43.25 | 2.7 | 0.58 |
| | | | carbonyl | | | | |
| 67 | 3-Cl-Ph | 3-Cl-Ph | 4-OH-pyridin-2- | >5 | >90 | >30 | 11.6 |
| | | | carbonyl | | | | |
| 68 | 3-Cl-4-Me-Ph | 3-Cl-4-Me-Ph | 4-CO₂H-pyridm-2- | 3.463 | 38.75 | 3 | 0.63 |
| | | | carbonyl | | | | |
| 69 | 4-Cl-2-Me-Ph | 4-Cl-2-Me-Ph | pyridin-2-carbonyl | 2.449 | 16.5 | 3.35 | 0.22 |
| 70 | 3-pyridyl | 3-Cl-Ph | 3-OH-pyridin-2- | >5 | 38.3 | 28.2 | 18.3 |
| | | | carbonyl | | | | |
| 71 | 3-Cl-phenyl | 3-[2- | 3-OH-pyridin-2- | >5 | 22 | 21.75 | 24.5 |
| | | (Me₂N)EtO]-Ph | carbonyl | | | | |
| 72 | 3-Cl-4-Me-Ph | 3-Cl-4-Me-Ph | 3-CO₂Et-pyridin-2- | 2.95 | 26.8 | 2.8 | 0.42 |
| | | | carbonyl | | | | |
| 73 | 3-Cl-4-Me-Ph | 3-Cl-4-Me-Ph | 3-CO₂Me-pyridin- | 2.997 | 21.3 | 7.2 | 0.63 |
| | | | 2-carbonyl | | | | |
| 74 | 4-Cl-Ph | 4-Cl-Ph | 3-(OSO₂NH₂)- | 3.912 | 24.45 | 8.2 | 0.71 |
| | | | pyridin-2-carbonyl | | | | |
| 75 | 3-Cl-Ph | 3-Cl-Ph | 3-[O(CH₂)₃CO₂H]- | 0.494 | 31.1 | 8.9 | 0.61 |
| | | | pyridin-2-carbonyl | | | | |
| 76 | 4-Cl-Ph | 4-Cl-Ph | 3-(2- | 3.81 | 19.8 | 4.05 | 2 |
| | | | morpholinoethyl) | | | | |
| | | | oxy-pyridin-2- | | | | |
| | | | carbonyl | | | | |
| 77 | 3-Cl-Ph | 3-Cl-Ph | 3-(2- | 1.225 | 17.2 | 5.15 | 1.1 |
| | | | morpholinoethyl) | | | | |
| | | | oxy-pyridin-2- | | | | |
| | | | carbonyl | | | | |
| 78 | 3-Cl-Ph | 3-Cl-Ph | 4-hydroxyquinolin- | >5 | >90 | >30 | 23.9 |
| | | | 2-yl | | | | |
| 79 | 3-Cl-4-Me-Ph | 3-Cl-4-Me-Ph | 4-methoxyquinolin- | 1.866 | 49.2 | 8.25 | 1.9 |
| | | | 2-yl | | | | |
| 80 | 4-Cl-Ph | 4-Cl-Ph | 2-Me-5, 7-di-Cl- | 1.71 | 1.06 | 1.315 | 0.12 |
| | | | quinolin-8-yl | | | | |
| 81 | 3-F-Ph | 3-DMISO-Ph | 5,7-di-Cl-quinolin- | 3.355 | 1.9 | 4.2 | 0.18 |
| | | | 8-yl | | | | |
| 82 | Pyridin-3-yl | vinyl | 2-Me-5, 7-di-Cl- | 2.647 | 1.19 | 1.95 | 0.22 |
| | | | quinolin-8-yl | | | | |
| 83 | 2-Cl-pyridin-4-yl | vinyl | 7-(Me₂N)-5-Cl- | 0.979 | 0.715 | 0.77 | 1.1 |
| | | | quinolin-8-yl | | | | |
| 84 | 3-(Me₂N)-Ph | Vinyl | 2-Me-5, 7-di-Cl- | 2.364 | 0.915 | 1.7 | 0.16 |
| | | | quinolin-8-yl | | | | |
| 85 | 3-Cl-Ph | 3-Cl-Ph | 4-OH-quinolin-2-yl | >5 | >90 | >30 | 23.9 |
| 86 | 3-Me-Ph | 3-Me-Ph | quinolin-8-yl | | 0.35 | 3 | 0.59 |
| 87 | 3-MeS-Ph | 3-MeS-Ph | quinolin-8-yl | 4.632 | 0.3 | 3.2 | 0.39 |
| 88 | 4-CN-Ph | 4-CN-Ph | quinolin-8-yl | | 0.41 | 1.385 | 0.76 |
| 89 | 4-Cl-3-F-Ph | 4-Cl-3-F-Ph | quinolin-8-yl | 1.385 | 0.41 | 1.36 | 0.38 |
| 90 | 4-Cl-Ph | 4-Cl-Ph | 5-Cl-7I-quinolin-8- | | 1.4 | 3.25 | 0.15 |
| | | | yl | | | | |
| 91 | 4-Cl-Ph | 4-F-Ph | 5,7-di-Me- | | 0.36 | 4.3 | 0.94 |
| | | | quinolin-8-yl | | | | |
| 92 | 4-Cl-Ph | 4-F-Ph | 5,7-di-Cl-quinolin- | | 1.1 | 2.55 | 0.052 |
| | | | 8-yl | | | | |
| 93 | 3-DMISO-Ph | Vinyl | quinolin-8-yl | 3.034 | 0.32 | 3.65 | 0.55 |
| 94 | Pyridin-3-yl | Vinyl | quinolin-8-yl | | 0.495 | 2.8 | 0.55 |
| 95 | 3-CN-Ph | 3,4-di-F-Ph | quinolin-8-yl | | 0.435 | 2.95 | 0.61 |
| 96 | 3-CN-Ph | 2,4-di-F-Ph | quinolin-8-yl | 3.035 | 0.34 | 3.3 | 0.63 |
| 97 | 4-CN-Ph | 3,4-di-F-Ph | quinolin-8-yl | 4.036 | 0.37 | 3.45 | 0.58 |
| 98 | 4-CN-Ph | 2,4-di-F-Ph | quinolin-8-yl | 3.245 | 0.345 | 4.05 | 0.59 |
| 99 | Pyridin-3-yl | 3-Cl-Ph | quinolin-8-yl | | 0.345 | 4.45 | 0.49 |
| 100 | 2-(MeO)- | Vinyl | quinolin-8-yl | | 0.305 | 3.8 | 0.33 |
| | pyridin-5-yl | | | | | | |
| 101 | 2-F-pyridin-5-yl | Vinyl | quinolin-8-yl | | 0.3 | 3.95 | 0.34 |
| 102 | pyridin-3-yl | 3-CN-Ph | quinolin-8-yl | | 0.49 | 4.2 | 0.37 |
| 103 | Furan-2-yl | 3-Cl-4-Me-Ph | quinolin-8-yl | | 0.38 | 4.4 | 0.35 |
| 104 | 4-[2- | 4-Cl-Ph | quinolin-8-yl | | 1.24 | 4.3 | 0.71 |
| | Me₂NCH₂CH₂ | | | | | | |
| | ]O-Ph | | | | | | |
| 105 | 4-(Me₂NCH₂)- | 4-Cl-Ph | quinolin-8-yl · HCl | | 0.41 | 4.2 | 0.72 |
| | Ph | | | | | | |
| 106 | 4-(Me₂NCH₂)- | 4-Cl-Ph | quinolin-8- | | 0.385 | 4.25 | 0.69 |
| | Ph | | yl-fumarate | | | | |
| 107 | 3-(MeS)-Ph | 3-Cl-Ph | quinolin-8-yl | | 0.365 | 4.1 | 0.42 |
| 108 | 4- | 3-Cl-Ph | quinolin-8-yl | 2.417 | 0.305 | 4.95 | 0.59 |
| | (MeSO₂NH)- | | | | | | |
| | Ph | | | | | | |
| 109 | 2-Me-4-Cl-Ph | 2-Me-4-Cl-Ph | quinolin-8-yl | | 0.405 | 4.35 | 0.75 |
| 110 | 4-(Me₂NCH₂)- | 3,4-di-F-Ph | quinolin-8-yl | | 0.38 | 4.7 | 0.61 |
| | Ph | | | | | | |
| 111 | 4-(4-Me- | 4-Cl-Ph | quinolin-8-yl | | 0.552 | 4.15 | 0.74 |
| | piperid-1yl- | | | | | | |
| | sulfonyl)-Ph | | | | | | |
| 112 | 4-(Me₂NCH₂)- | 3-CN-Ph | quinolin-8- | | 0.44 | 4.2 | 0.98 |
| | Ph | | yl-fumarate | | | | |
| 113 | 3-CN-Ph | 2,5-di-F-Ph | quinolin-8-yl | | 0.39 | 4.25 | 0.57 |
| 114 | 3,4-di-F-Ph | 4-Cl-Ph | quinolin-8-yl | | 0.41 | 4 | 0.71 |
| 115 | 3-F-Ph | 4-Cl-3- | quinolin-8-yl | 3.077 | 0.465 | 3.6 | 0.59 |
| | | CF₃-Ph | | | | | |
| 116 | 3-Cl-4-F-Ph | Vinyl | quinolin-8-yl | 3.95 | 0.5865 | 3.65 | >30 |
| 117 | furan-2-yl | Vinyl | quinolin-8-yl | | 0.295 | 3.65 | 0.63 |
| 118 | 3,5-di-Me- | Vinyl | quinolin-8-yl | | 0.265 | 3.5 | 0.44 |
| | isoxazol-4-yl | | | | | | |
| 119 | 4-(Me₂NCH₂)- | Vinyl | quinolin-8-yl | | 0.51 | 3.8 | 0.36 |
| | Ph | | | | | | |
| 120 | 4-(Me₂NCH₂)- | 3-F-5-CF₃- | quinolin-8-yl | 2.794 | 0.42 | 4.9 | 0.44 |
| | Ph | Ph | | | | | |
| 121 | 3-CN-Ph | 3-F-5-CF₃-Ph | quinolin-8-yl | | 0.43 | 4.95 | 0.53 |
| 122 | 4-CN-Ph | 3-F-5-CF₃-Ph | quinolin-8-yl | 4.577 | 0.4 | 4.55 | 0.45 |
| 123 | 3-Cl-Ph | 3-F-5-CF₃-Ph | quinolin-8-yl | 3.51 | 0.31 | 4 | 0.54 |
| 124 | 4-(Me₂NCH₂)-Ph | Vinyl | quinolin-8-yl · HCl | | 0.445 | 3.95 | 0.3 |
| 125 | 4-(Me₂NCH₂)-Ph | 4-CN-Ph | quinolin-8-yl | 4.973 | 0.5 | 4.9 | 0.38 |
| 126 | 4-(Me₂NCH₂)-Ph | 3-DMISO-Ph | quinolin-8-yl | 4.746 | 0.595 | 5.05 | 0.45 |
| 127 | 3-Cl-4-Me-Ph | 3-Cl-4-Me-Ph | 3-OH-pyridin-2- | | 2.5 | 6.3 | 0.36 |
| | | | carbonyl | | | | |
| 128 | 3-Cl-4-Me-Ph | 3-Cl-4-Me-Ph | 3-CO₂H-pyridin-2- | | 8 | 1.75 | 0.61 |
| | | | carbonyl | | | | |
| 129 | 3-Cl-4-Me-Ph | 3-Cl-4-Me-Ph | 3-NH₂-pyridin-2- | | 4.4 | 0.855 | 1.6 |
| | | | carbonyl | | | | |
| 130 | 3-Cl-4-Me-Ph | 3-Cl-4-Me-Ph | 5,7-di-Cl-4-OH- | | 18.1 | >30 | 13 |
| | | | quinolin-2-yl | | | | |
| 131 | 4-Cl-Ph | 4-Cl-Ph | 6-NH₂-pyridin-2- | 0.6855 | 7.6 | 0.0535 | 0.24 |
| | | | carbonyl | | | | |
| 132 | 4-Cl-Ph | 4-Cl-Ph | 6-OH-pyridin-2- | | 45.7 | 23.4 | 14.1 |
| | | | carbonyl | | | | |
| 133 | Pyridin-3-yl | 3-CN-Ph | 3-OH-pyridin-2- | | 0.86 | 10.4 | 2.2 |
| | | | carbonyl | | | | |
| 134 | 4-Cl-2-Me-Ph | 4-Cl-2-Me-Ph | 3-OH-pyridin-2- | | 1.7 | 3.6 | 0.12 |
| | | | carbonyl | | | | |

CMPD is the compound number used in the examples below, and it refers to the numbered structures in the application. IC₅₀ μg/ml is inhibitory concentration in micrograms per milliliter. R* and R** refer to the substituents attached to the boron atom as depicted in the formulas. LIGAND refers to the ring structure bound to the boron atom in the formulas and making up the ring that contains the boron atom.

TABLE 2
In vivo Efficacy of Selected Compounds
in Mouse Model Against P. Berghei

| | | | Parasitized RBC over 100 | | | Mean |
|---|---|---|---|---|---|---|
| Com- | Dosage | | | % of | % of | Survival |
| pound | mg/kg | Route | Mean | Control | Activity | (Days) |
| 11 | 4 × 30 | s.c., 1×/day | 14.3 | 57.54 | 42.46 | 19.7 |
| 13 | 4 × 30 | s.c., 2×/day | 11.9 | 47.09 | 52.31 | 15.3 |
| 27 | 4 × 100 | s.c., 2×/day | 7.9 | 31.91 | 68.09 | 18.7 |
| 11 | 4 × 100 | p.o., 1×/day | 10.4 | 41.78 | 58.22 | 15.0 |
| 13 | 4 × 100 | p.o., 2×/day | 4.7 | 18.78 | 81.29 | 12.7 |
| 27 | 4 × 300 | p.o., 2×/day | 10.7 | 43.08 | 56.94 | 15.7 |
| Control | | | 24.9 | | | 6.3 |

The compound number refers to the numbered structures in the application; mg/kg is the number of milligrams of compound administered per kilogram of body weight of the mouse.

The present invention also encompasses the anti-parasitic use of the acylated prodrugs of the compounds described herein. Those skilled in the art will recognize various synthetic methodologies which may be employed to prepare non-toxic, pharmaceutically acceptable addition salts and acylated prodrug compounds for the treatment of parasitic infections.

### 5.9 Synthetic Examples

### 5.9.1 General

Proton NMR are recorded on Varian AS 400 spectrometer and chemical shifts are reported as δ (ppm) down field from tetramethylsilane. Mass spectra are determined on Micromass Quattro II and Applied Biosystem AP3000. Compound identification numbers appear in parentheses and some of them correspond to the numbers in Scheme 1, Table 1 and Table 2.

### 5.9.2 Formation of Ethylene Glycol Boronate Ester (3, T=single bond) General Procedure

Boronic acid was dissolved in dry THF or dry diethyl ether (˜10 mL/g) under nitrogen. Ethylene glycol (1 molar equivalent) was added to the reaction and the reaction was heated to reflux for 1- to 4 hours. Reaction was cooled to room temperature and solvent was removed under reduced pressure leaving the ethylene glycol ester as an oil or a solid. In cases where an oil was obtained or a solid that dissolved in hexane, dry hexane was added and removed under reduced pressure. The product was then placed under high vacuum for several hours. In cases where a solid was obtained that did not dissolve in hexane, the solid was collected by filtration and washed with cold hexane.

### 5.9.2.1 3-Cyanophenylboronic acid ethylene glycol ester (3a)

3-Cyanophenylboronic acid (1 g, 6.8 mmol) was dissolved in dry THF (10 mL) under nitrogen. Ethylene glycol (379 μL, 422 mg, 6.8 mmol) was added and the reaction was heated to reflux for 4 hours then cooled to room temperature. THF was removed by rotary evaporator to give a white solid. Cold hexane was added and the product was collected by filtration giving a white solid (1.18 g, quant. yield). ¹H-NMR (300.058 MHz, DMSO-d6) δ ppm 7.92-8.01 (3H, m), 7.50-7.64 (1H, m), 4.35 (4H, s)

### 5.9.2.2 Thiophene 3-boronic acid ethylene glycol ester (3b)

Thiophene-3-boronic acid (1 g, 7.8 mmol) was dissolved in dry THF (10 mL) under nitrogen. Ethylene glycol (435 μL, 484 mg, 7.8 mmol) was added and the reaction was heated to reflux for 1 hour then cooled to room temperature. THF was removed by rotary evaporator to give a white solid. Hexane was added, dissolving the solid and removed by rotary evaporation. The product was placed under high vacuum to yield a tan solid (1.17 g, 97%). ¹H-NMR (300.058 MHz, CDCl3) δ ppm 7.93 (1H, s), 7.3-7.4 (2H, m), 4.35 (4H, s).

### 5.9.3 Formation of Unsymmetrical Borinic Acid (6) From Boronic Acid Ethylene Glycol Ester General Procedure A: Grignard Methodology

Boronic acid ethylene glycol ester was dissolved in dry THF (10-20 ml/g) under nitrogen. Solution was cooled to −78° C. in an acetone-dry ice bath or to 0° C. in an ice-water bath. Grignard reagent (0.95 to 1.2 molar equivalent) was added drop wise to the cooled solution. The reaction was warmed to room temperature and stirred for 3-18 hours. 6N HCl (2 mL/g) was added and solvent was removed under reduced vacuum. Product was extracted into diethyl ether (40 mL/g) and washed with water (3× equal volume). Organic layer was dried (MgSO₄), filtered and the solvent was removed by rotary evaporation giving the crude product, which is either purified by column chromatography or taken onto the next step without purification. Alternative work-up: if the borinic acid product contained a basic group such as an amine or pyridine, then after stirring at room temperature for 3-18 hours, water (2 mL/g) was added and the pH adjusted to 8. Product was extracted into diethyl ether or ethylacetate or THF up to three times (40 mL/g). Organic layer was dried (MgSO₄), filtered and the solvent was removed by rotary evaporation giving the crude product, which is either purified by column chromatography or taken onto the next step without purification.

### 5.9.3.1 (4-Cyanophenyl)(3-fluorophenyl)borinic acid (6a)

4-Cyanophenyl boronic acid ethylene glycol ester (500 mg, 2.89 mmol) was dissolved in dry THF under nitrogen. The solution was cooled to −78° C. in an acetone/dry ice bath and 3-fluorophenylmagnesium bromide (1M in THF)(2.74 mL, 2.74 mmol, 0.95 molar equivalent) was added drop wise to the cold solution. The reaction was allowed to warm slowly to room temperature and stirred for 18 hours. 6N HCl (1 mL) was added to the reaction causing a cloudy appearance and the solvent was removed using a rotary evaporator. The product was extracted into diethyl ether (20 mL) and washed with water (3×20 mL). The organic layer was dried (MgSO₄), filtered and the solvent removed using a rotary evaporator to yield the crude product as an oily solid. This was taken onto the next step without purification.

### 5.9.4 General Procedure B: (Hetero)aryl Lithium Methodology

The (hetero)aryl-bromide or iodide was dissolved in dry THF (20-30 mL/g) under nitrogen and degassed. The solution was cooled to −78° C. in an acetone-dry ice bath and n-, sec- or tert-butyllithium in THF or other solvent (1.2-2.4 molar equivalents) was added to the cooled solution drop wise generally causing the solution to turn deep yellow. The boronic acid ethylene glycol ester (1 molar equivalent) was dissolved in dry THF or diethyl ether (2-10 mL/g) under nitrogen. The boronic acid ethylene glycol ester in THF was added drop wise to the cooled aryl-lithium solution generally causing the solution to turn pale yellow. The reaction was warmed to room temperature and stirred for 1-18 hours. 6N HCl (2-4 mL/g) was added and solvent was removed under reduced vacuum. Product was extracted into diethyl ether (40 mL/g) and washed with water (3× equal volume). Organic layer was dried (MgSO₄), filtered and the solvent was removed by rotary evaporation giving the crude product, which is either purified by column chromatography or taken onto the next step without purification. Alternative work-up: if the borinic acid product contained a basic group such as an amine or pyridine then after stirring at room temperature for 3-18 hours water (2 mL/g) was added and the pH adjusted to 5-7. Product was extracted into diethyl ether or ethylacetate or THF (40 mL/g) and washed with water (3× equal volume). Organic layer was dried (MgSO₄), filtered and the solvent was removed by rotary evaporation giving the crude product, which is either purified by column chromatography or taken onto the next step without purification.

### 5.9.4.1 (3-Thienyl)(3-chlorophenyl)borinic acid (6b)

3-Chloro-bromobenzene (447 μL, 728 mg, 3.8 mmol) was dissolved in dry THF (15 mL) under nitrogen. The solution was degassed and cooled to −78° C. in an acetone-dry ice bath. tert-Butyllithium (1.7 M in THF, 4.47 mL, 7.6 mmol, 2 molar equivalent) was added to the cooled solution drop wise causing the solution to turn deep yellow. The solution was stirred at −78° C. while 3-thiopheneboronic acid ethylene glycol ester (586 mg) was dissolved in dry diethyl ether (1 mL). The boronic ester solution was then added drop wise to the cooled solution causing the color to change to pale yellow. The reaction was warmed to room temperature and stirred for 18 hours. 6N HCl (2 mL) was added and the reaction was stirred for 1 hour. The solvent was removed using a rotary evaporator. The product was extracted into diethyl ether (10 mL) and washed with water (2×10 mL). The organic layer was dried (MgSO₄), filtered and the solvent removed using a rotary evaporator to yield the crude product as an orange oil. The product was purified by column chromatography using silica gel and hexane:ethyl acetate 5:1 as eluent giving the pure product as a clear oil (614 mg, 73%).

### 5.9.4.2 (3-Chlorophenyl)vinylborinic acid (6c)

This was prepared by a similar process as described for 6b by the reaction of 3-cyanophenyl boronic acid ethylene glycol ester with Vinylmagnesium bromide.

### 5.9.4.3 (3-Fluoro-5-chlorophenyl)ethynylborinic acid (6d)

This was prepared by a similar process as described for 6b by the reaction of 3-fluoro-5-chlorophenyl boronic acid ethylene glycol ester with ethynylmagnesium bromide.

### 5.9.4.4 (4-Methyl-3-chlorophenyl)(2-thienyl)borinic acid (6e)

This was prepared by a similar process as described for 6b by the reaction of 2-thienylboronic acid ethylene glycol ester with 4-methyl-3-chlorophenyltithium.

### 5.9.4.5 (4-Cyanophenyl)ethynylborinic acid (6f)

This was prepared by a similar process as described for 6b by the reaction of 4-cyanophenylboronic acid ethylene glycol ester with ethynylmagnesium bromide.

### 5.9.4.6 (3-Fluorophenyl)Cyclopropylborinic acid (6g)

This was prepared by a similar process as described for 6b by the reaction of 3-fluorophenylboronic acid ethylene glycol ester with cyclopropyllithium.

### 5.9.4.7 (3-Thienyl)methylborinic acid (6h)

This was prepared by a similar process as described for 6b by the reaction of 3-thienylboronic acid ethylene glycol ester with methyllithium.

### 5.9.4.8 (4-Pyridyl)phenylborinic acid (6i)

This was prepared by a similar process as described for 6b by the reaction of phenylboronic acid ethylene glycol ester with 4-pyridyllithium.

### 5.9.4.9 (3-Cyanophenyl)(2-fluorophenyl)borinic acid (6j)

This was prepared by a similar process as described for 6b by the reaction of 3-cyanophenylboronic acid ethylene glycol ester with 2-fluorophenyllithium.

### 5.9.5 Formation of Symmetrical Borinic Acid (5) By Reaction Of Organometallics With Trialkyl Borates: Bis(4-Chlorophenyl)Borinic Acid (5a) (Procedure C)

A cold solution (−78° C.) of trimethyl borate (0.37 mL) in dry tetrahydrofuran (THF, 25 mL) was treated drop wise with 4-chlorophenylmagnesium bromide (6.75 mL, 1M solution in ether). The reaction mixture was stirred at −78° C. for 1 h and then stirred for 18 h at room temperature. The solvent was removed under reduced pressure. The resultant residue was stirred with 100 mL of ether and 15 mL of 6N hydrochloric acid. Organic layer was separated and aqueous layer was extracted with ether (2×100 mL). The combined organic extract was washed with brine and dried over anhydrous magnesium sulfate. Solvent was removed to give light yellowish solid. The product was chromatographed over silica gel (Hex:Ether=1:1) to give 420 mg of borinic acid. ¹H NMR (400 MHz, CDCl₃) δ: 5.84 (s, OH), 7.46 (d, 4H, Ar—H), 7.72 (d, 4H, Ar—H).

### 5.9.5.1 Bis(3-chloro-4-methylphenyl)borinic acid (5b)

In a similar manner as for 5a, the titled compound was obtained from the reaction of 3-chloro-4-methylphenylmagnesium bromide with trimethyl borate. The product was obtained by chromatography over silica gel.

### 5.9.5.2 Bis(3-fluoro-4-methylphenyl)borinic acid (5c)

In a similar manner as for 5a, the titled compound was obtained from the reaction of 3-fluoro-4-methylphenyllithium with trimethyl borate. The product was obtained by chromatography over silica gel.

### 5.9.5.3 Bis(3-chloro-4-methoxyphenyl)borinic acid (5d)

In a similar manner as for 5a, the titled compound was obtained from the reaction of 3-chloro-4-methoxyphenyllithium with trimethyl borate. The product was obtained by chromatography over silica gel.

### 5.9.5.4 Bis(3-fluoro-4-methoxyphenyl)borinic acid (5e)

In a similar manner as for 5a, the titled compound was obtained from the 25 reaction of3-fluoro-4-methoxyphenyllithium with trimethyl borate. The product was obtained by chromatography over silica gel.

### 5.9.6 Formation of Unsymmetrical borinic acids (6) by Reaction of organometallics with alkyl(aryl)dialkoxyboranes. (4-Chlorophenyl)methylborinic acid (6k) (Procedure D)

To 4-chlorophenylmagnesium bromide (5.5 mL, 1M solution in ether) at −78° C., di(isopropoxy)methylborane (1 mL, 0.78 g) was added drop wise via syringe. The reaction mixture was stirred at −78° C. for 1 h and then stirred overnight at ambient temperature. The reaction mixture was treated drop wise with 100 mL of ether and 15 mL of 6N hydrochloric acid, and stirred for 1 h. Organic layer was separated and aqueous layer was extracted with ether (2×100 mL). The combined organic extract was washed with brine and dried over anhydrous sodium sulfate. Solvent was removed under reduce pressure to give 1.1 g of oil. ¹H NMR of the product was consistent for (4-chlorophenyl)methyl borinic acid.

### 5.9.6.1 (4-Fluorophenyl)methylborinic acid (6m)

In a similar manner as for 6k, the titled compound was obtained from the reaction of 4-fluorophenylmagnesium bromide with di(isopropoxy)methylborane. The product was obtained by chromatography over silica gel.

### 5.9.6.2 (4-Biphenyl)methylborinic acid (6n)

In a similar manner as for 6k, the titled compound was obtained from the 20 reaction of 4-biphenyllithium with di(isopropoxy)methylborane. The product was obtained by chromatography over silica gel.

### 5.9.6.3 (3-Chloro-4-methylphenyl)methylborinic acid (6o)

In a similar manner as for 6k, the titled compound was obtained from the reaction of 3-chloro-4-methylphenyllithium with di(isopropoxy)methylborane. The product was obtained by chromatography over silica gel.

### 5.9.6.4 (3-Chloro-4-methoxyphenyl)methylborinic acid (6p)

In a similar manner as for 6k, the titled compound was obtained from the reaction of 3-chloro-4-methoxyphenyllithium with di(isopropoxy)methylborane. The product was obtained by chromatography over silica gel.

### 5.9.6.5 (4-Dimethylaminophenyl)methylborinic acid (6q)

In a similar manner as for 6k, the titled compound was obtained from the reaction of 4-dimethylaminophenyllithium with di(isopropoxy)methylborane. The product was obtained by chromatography over silica gel.

### 5.9.6.6 (3-Chloro-4-dimethylaminophenyl)vinylborinic acid (6r)

In a similar manner as for 6k, the titled compound was obtained from the reaction of 3-chloro-4-dimethylaminophenyllithium with di(butoxy)vinylborane. The product was obtained by chromatography over silica gel.

### 5.9.6.7 Pyridylvinyl borinic acid (6s)

To a solution of 3-bromopyridine (1.60 g, 10.0 mmol) in THF (15 mL) was added isopropylmagnesium chloride (2.0 M in THF) (5.0 mL, 10 mmol) under nitrogen atmosphere at room temperature, and the mixture was stirred for 1 h. To the mixture was added vinylboronic acid dibutyl ester (3.4 mL) drop wise, and the mixture was stirred at room temperature for 18 h. Water was added and the pH was adjusted to 7 with 1 M hydrochloric acid. The mixture was extracted with ethyl acetate. The organic layer was washed with brine and dried on anhydrous sodium sulfate. The solvent was removed under reduced pressure to give the title compound (1.04 g, 78%).

### 5.9.6.8 Bis(3-Chlorophenyl)borinic acid 4-(hydroxyethyl)imidazole ester (60)

To a solution of bis(3-chlorophenyl)borinic acid (0.4 g, 1.428 mmol) in ethanol (10 mL), 4-(hydroxyethyl)imidazole hydrochloride (0.191 g, 1.428 mmol), sodium bicarbonate (0.180 g, 2.143 mmol) were added and the reaction mixture was stirred at room temperature for 18 h. Salt was removed by filtration. Filtrate was concentrated and treated with hexane to afford the product as a solid and was collected by filtration. (450 mg, 84.9% yield). MS (ESI−): m/z=343 (M⁻−1).

### 5.9.6.9 Bis(4-Chlorophenyl)borinic acid 4-(hydroxymethyl)imidazole ester (61)

In a similar manner as in Example 60, the titled compound was obtained from the reaction of bis(4-chlorophenyl)borinic acid with 4-(hydroxymethyl)-imidazole hydrochloride. The product was obtained as white crystals. MS (ESI−): m/z=329 (M⁻−1).

### 5.9.6.10 Bis(3-chloro-4-methylphenyl)borinic acid 1-benzyl-4-(hydroxymethyl) imidazole ester (62)

To a solution of 1-benzyl-4-(hydroxymethyl)imidazole (96 mg, 0.521 mmol) in methanol (5 mL), bis(3-chloro-4-methylphenyl)borinic acid (121 mg, 0.521 mmol) was added and the reaction mixture was stirred at room temperature for 2 h. Solvent was removed under reduced pressure and the residue was treated with hexane to give a solid. The product was isolated by filtration and washed with hexane to give product (193 mg, 83%). ¹H NMR (CDCl₃) δ: 2.3 (s, 6H, 2×CH3), 4.8 (brs, 2H, CH2), 5.1 (brs, 2H, CH2), 6.9-7.4 (complex, 13H, Ar—H); MS (ES+)(m/z) 448.78, MF C₂₅H₂₃BCl₂N₂O.

### 5.9.6.11 Bis(3-chloro-4-methylphenyl)borinic acid 1-methyl-2-(hydroxymethyl) imidazole ester (63)

In a similar manner as in Section 5.9.6.10, the titled compound was obtained from the reaction of bis(3-chloro-4-methylphenyl)borinic acid with 1-methyl-2(hydroxymethyl)imidazole hydrochloride. The product was obtained as white crystals. MS (ESI+): m/z=373 (M⁺−1).

### 5.9.6.12 Bis(3-chloro-4-methylphenyl)borinic acid 1-ethyl-2-(hydroxymethyl)imidazole ester (64)

In a similar manner as in Section 5.9.6.10, the titled compound was obtained from the reaction of bis(3-chloro-4-methylphenyl)borinic acid with 1-ethyl-2-(hydroxymethyl)imidazole hydrochloride. The product was obtained as white crystals. MS (ESI+): m/z=387 (M⁺−1).

### 5.9.6.13 Bis(3-chloro-4-methylphenyl)borinic acid 1-methyl-4-(hydroxymethyl) imidazole ester (65)

In a similar manner as in Section 5.9.6.10, the titled compound was obtained from the reaction of bis(3 -chloro-4-methylphenyl)borinic acid with 1-methyl-4-(hydroxymethyl)imidazole hydrochloride. The product was obtained as white crystals. MS (ESI+): m/z=373 (M⁺−1).

### 5.9.6.14 Bis(3-chloro-4-methylphenyl)borinic acid 2-pyridylethanol ester (66)

In a similar manner as in Section 5.9.6.8, the titled compound was obtained from the reaction of bis(3-chloro-4-methylphenyl)borinic acid with 2-pyridylethanol. The product was obtained as white crystals. MS (ESI+): m/z=384 (M⁺).

### 5.9.6.15 Bis(4-chlorophenyl)borinic acid 2-pyridylmethanol ester (67)

In a similar manner as in Section 5.9.6.8, the titled compound was obtained from the reaction of bis(4-chlorophenyl)borinic acid with 2-pyridylmethanol. The product was obtained as white crystals. MS (ESI+): m/z=342 (M⁺+1).

### 5.9.6.16 Bis(4-fluorophenyl)borinic acid 2-pyridylmethanol ester (68)

In a similar manner as in Section 5.9.6.8, the titled compound was obtained from the reaction of bis(4-fluorophenyl)borinic acid with 2-pyridylmethanol. The 5 product was obtained as white crystals. ¹H NMR (CDCl₃): δ=5.3 (s, 2H), 6.9 (t, 4H), 7.3 (t, 4H), 7.5-7.6 (m, 2H), 8.1 (t, 1H) and 8.3 (d, 1H) ppm.

### 5.9.7 Hydroxyquinoline Derivatives

### 5.9.7.1 Bis(3-chlorophenyl)borinic acid 5-cyano-quinolin-8-yl ester (16)

A solution of bis(3-chlorophenyl)borinic acid (0.25 g) in ethanol (10 mL) was mixed with a solution of 5-cyano-8-hydroxquinoline (0.15 g) in ethanol (5 mL) and water (2 mL). The mixture was stirred at 5° C. The reaction mixture was then stirred at ambient temperature, and a yellow solid precipitate formed. The reaction mixture was stirred for additional 21 hours. The product was isolated by filtration, washed with hexane and air dried to give 272 mg of complex. MS: m/z=171 (ESI+); m/z=251, 249 and 169 (ESI−).

### 5.9.7.2 Bis(3-chloro-4-fluoro-phenyl)borinic acid quinolin-8-yl ester (12)

In a similar manner as in Section 5.9.7.1, the titled compound was obtained from the reaction of bis(3-chloro-4-fluorophenyl)borinic acid with 8-hydroxyquinoline. The product was obtained as yellow crystals. MS (ESI−): m/z=287 and 285.

### 5.9.7.3 (4-Chlorophenyl)(3-fluorophenyl) borinic acid quinolin-8-yl ester (11)

In a similar manner as in Section 5.9.7.1, the titled compound was obtained 30 from the reaction of (3-fluorophenyl)(4-chlorophenyl)borinic acid with 8-hydroxyquinoline. The product was obtained as yellow crystals. MS: m/z=250 (ESI+); m/z=235 and 233 (ESI−).

### 5.9.7.4 (4-chlorophenyl)(4-fluorophenyl) borinic acid quinolin-8-yl ester (15)

In a similar manner as in Section 5.9.7.1, the titled compound was obtained from the reaction of (4-fluorophenyl)(4-chlorophenyl)borinic acid with 8-hydroxyquinoline. The product was obtained as yellow crystals. MS: m/z=146 (ESI+); m/z=235 and 233 (ESI−).

### 5.9.7.5 (3-Pyridyl)vinylborinic acid 8-hydroxyquinoline ester (49)

A mixture of (3-pyridyl)vinylborinic acid (1.04 g) and 8-hydroxyquinoline (0.961 g) in ethanol 30 mL was stirred at 40° C. for 20 min. The solvent was removed under reduced pressure and the residue was treated with diethyl ether/diisopropyl ether/hexane to afford the desired complex as yellow crystals. H NMR (300 MHz, DMSO-d6) δ (ppm) 5.23 (dd, J=19.3, 4.1 Hz, 1H), 5.46 (dd, J=13.5, 4.1 Hz, 1H), 6.43 (dd, J=19.3, 13.5 Hz, 1H), 7.14 (d, J=7.6 Hz, 1H), 7.19 (dd, J=7.6, 4.7 Hz, 1 H), 7.41 (d, J=8.2 Hz, 1H), 7.6-7.8 (m, 2H), 7.88 (dd, J=8.5, 5.0 Hz, 1H), 8.35 (dd, J=5.0, 2.1 Hz, 1H), 8.57 (s, 1H), 8.76 (d, J=8.5 Hz, 1H), 9.00 (d, J=5.0 Hz, 1H) ESI-MS m/z 261 (positive); C₁₆H₁₃BN₂O=260.11

### 5.9.7.6 (2-Thienylmethylborinic acid quinolin-8-yl ester(51)

In a similar manner as in Section 5.9.7.1, the titled compound was obtained from the reaction of (2-thienyl)methylborinic acid with 8-hydroxyquinoline. The product was obtained a yellow crystals.

### 5.9.7.7 (3-Chlorophenyl)(2-thienyl)borinic acid quinolin-8-yl ester(52)

In a similar manner as in Section 5.9.7.1, the titled compound was obtained 30 from the reaction of (3-chlorophenyl)(2-thienyl)borinic acid with 8-hydroxyquinoline. The product was obtained as yellow crystals. MS (ESI+): m/z=350 (M⁺+1).

### 5.9.7.8 (3-Cyanophenyl)vinylborinic acid quinolin-8-yl ester (37)

In a similar manner as in Section 5.9.7.1, the titled compound was obtained 5 from the reaction of (3-cyanophenyl)vinylborinic acid with 8-hydroxyquinoline. The product was obtained as yellow crystals. MS (ESI+): m/z=285 (M⁺+1).

### 5.9.7.9 (2-Chlorophenyl)ethynylborinic acid quinolin-8-yl ester (53)

In a similar manner as in Section 5.9.7.1, the titled compound was obtained from the reaction of (2-chlorophenyl)ethynylborinic acid with 8-hydroxyquinoline. The product was obtained as yellow crystals. MS (ESI, positive): m/z=291 (M+) and 292 (M+1); ¹H NMR (DMSO-d6, 300 MHz): [symbol] 8.82 (d, 1H), 8.78 (d, 1H), 8.03 (dd, 1H), 7.88 (dd, 1H), 7.70 (t, 1H), 7.46 (d, 1H), 7.33-7.24 (m, 2H), 7.18 (dd, 1H), 7.10 (d, 1H) and 3.04 (s, 1H) ppm.

### 5.9.7.10 Bis(ethynyl)borinic acid 8-Hydroxyquinoline ester (54)

In a similar manner as in Section 5.9.7.1, the titled compound was obtained from the reaction of bis(ethynyl)borinic acid THE solution with 8-hydroxyquinoline. Bis(ethynyl)borinic acid was prepared from ethynylmagnesium bromide and trimethyl borate without rotary evaporation of THF during its work-up process because this borinic acid is very volatile. The complex product was obtained as light yellow crystals. MS (ESI, positive): m/z=205 (M+) and 206 (M+1); ′H NMR (DMSO-d6, 300 MHz): [symbol] 9.05 (dd, 1H), 8.84 (dd, 1H), 7.97 (dd, 1H), 7.68 (t, 1H), 7.70 (d, 1H), 7.08 (d, 1H) and 2.90 (s, 2H) ppm.

### 5.9.7.11 (3-Fluorophenyl)cyclopropylborinic acid 8-hydroxyquinoline ester (55)

In a similar manner as in Section 5.9.7.1, the titled compound was obtained from the reaction of (3-fluorophenyl)cyclopropylborinic acid with 8-hydroxyquinoline. The product was obtained as light yellow crystals. ¹H NMR (DMSO-d₆): δ=0.25-0.20 (m, 1H), 0.10-0.25 (m, 3H), 0.3-0.4 (m, 1H), 6.9-7.0 (m, 1H), 7.1 (d, 1H), 7.2-7.3 (m, 3H), 7.4 (d, 1H), 7.65 (t, 1H), 7.9 (dd, 1H), 8.75 (d, 1H) and 9.1 (d, 1H) ppm.

### 5.9.7.12 Divinylborinic acid quinolin-8-yl ester (70)

The title compound was prepared by the procedure described in Section 5.9.7.1 and the compound was obtained as yellow crystals. MS (ESI, positive): m/z=209 (M+) and 210 (M+1); ¹H NMR (DMSO-d6, 300 MHz): [symbol] 8.75-8.65 (m, 2H), 7.87 (dd, 1H), 7.63 (t, 1H), 7.34 (d, 1H), 7.02 (d, 1H), 6.17 (dd, 2H), 5.36 (dd, 2H) and 5.20 (dd, 2H) ppm.

### 5.9.7.13 (3-Chlorophenyl)(3,4-dimethoxyphenvi)borinic acid 8-hydroxyquinoline ester (71)

(3-Chlorophenyl)(3,4-dimethoxyphenyl)borinic acid was prepared from 3,4-dimethoxyphenylmagnesium bromide and 3-chlorophenylboronic acid ethylene glycol ester by the procedure described in Example 6a. The title complex product was made by the methodology described in Section 5.9.7.1, and obtained as yellow crystals. MS (ESI, positive): m/z=404 (M+1); ¹H NMR (DMSO-d6, 300 MHz): [symbol] 9.17 (d, 1H), 8.78 (d, 1H), 7.90 (dd, 1H), 7.70 (t, 1H), 7.43 (d, 1H), 7.30-7.17 (m, 5H), 6.89-6.80 (m, 3H), 3,66 (s, 3H) and 3.62 (s, 3H) ppm.

### 5.9.7.14 (2-Chlorophenyl)vinylborinic acid 8-hydroxyquinoline ester (72)

In a similar manner as in Section 5.9.7.1, the titled compound was obtained from the reaction of (2-chlorophenyl)(vinyl)borinic acid with 8-hydroxyquinoline. The product was obtained as yellow crystals. MS (ESI, positive): m/z=293 (M+) and 294 (M+1); ¹H NMR (DMSO-d6, 300 MHz): [symbol] 8.80 (d, 1H), 8.75 (d, 1H),0.84(dd, 1H), 7.65 (t, 1H), 7.55-7.50 (m, 1H), 7.38 (d, 1H), 7.20-7.16 (m, 3H), 7.08 (d, 1H), 6.54 (dd, 1H), 5.40 (dd, 1H) and 5.12 (dd, 1H) ppm.

### 5.9.7.15 (3-Fluorophenyl)vinylborinic acid quinolin-8-yl ester (73)

In a similar manner as in Section 5.9.7.1, the titled compound was obtained from the reaction of (3-fluorophenyl)(vinyl)borinic acid with 8-hydroxyquinoline. The product was obtained as yellow crystals. MS (ESI, positive): m/z=277 (M+) and 278 (M+1); ¹H NMR (DMSO-d6, 300 MHz): δ 8.80 (d, 1H), 8.74 (d, 1H), 7.87 5 (dd, 1H), 7.67 (t, 1H), 7.40 (d, 1H), 7.28-7.20 (m, 2H), 7.14-7.11 (m, 2H), 6.97-6.90 (m, 1H), 6.41 (dd, 1 H), 5.44 (dd, 1H) and 5.21 (dd, 1H) ppm.

### 5.9.7.16 (3-Chlorophenyl)ethynylborinic acid 8-Hydroxyquinoline ester (74)

In a similar manner as in Section 5.9.7.1, the titled compound was obtained from the reaction of (3-chlorophenyl)ethynylborinic acid with 8-hydroxyquinoline. The product was obtained as yellow crystals. MS (ESI, positive): m/z=291 (M+) and 292 (M+1); ¹H NMR (DMSO-d6, 300 MHz): δ 8.93 (d, 1H), 8.80 (d, 1H), 7.89 (dd, 1H), 7.71 (t, 1H), 7.47 (d, 1H), 7.45 (d, 1H), 7.35-7.31 (m, 1H), 7.25-7.22 (m, 15 2H), 7.18 (d, 1H) and 3.05 (s, 1H) ppm.

### 5.9.7.17 (3-Chloro-4-methylphenyl)phenylborinic acid quinolin-8-yl ester (10)

In a similar manner as in Section 5.9.7.1, the titled compound was prepared from (3-chloro-4-methylphenyl)phenylborinic acid and 8-hydroxyquinoline to afford a yellow crystalline solid. ESI-MS m/z 358 (M+H)⁺, C₂₂H₁₇B³⁵ClNO=357.

### 5.9.7.18 Bis[3-(4,5-dihydro-4,4-dimethyloxazol-2-yl)phenyl]borinic acid 8-hydroxyquinoline ester (13)

In a similar manner as in Section 5.9.7.1, the titled compound was prepared from bis[3-(4,5-duhydro-4,4-dimethyloxazol-2yl)phenyl]borinic acid and 8-hydroxyquinoline to afford a yellow crystalline solid. ESI-MS m/z 504 (M+H)⁺, C₃₁H₃₀BN₃O₃=503.

### 5.9.7.19 (3-Chlorophenyl)(4-fluorophenyl)borinic acid quinolin-8-yl ester (14)

In a manner as in Section 5.9.7.1, the titled compound was prepared from (3-chlorophenyl)(4-fluorophenyl)borinic acid and 8-hydroxyquinoline to afford a yellow crystalline solid. ESI-MS m/z 362 (M+H)⁺, C₂₁H₁₄B³⁵ClFNO=361.

### 5.9.7.20 (3-Chlorophenyl)(3-fluorophenyl)borinic acid quinolin-8-yl ester (17)

In a manner as in Section 5.9.7.1, the titled compound was prepared from (3-chlorophenyl)(3-fluorophenyl)borinic acid and 8-hydroxyquinoline to afford a yellow crystalline solid. ESI-MS m/z 362 (M+H)⁺, C₂₁H₁₄B³⁵ClFNO=361.

### 5.9.7.21 (3-Chlorophenyl)[3-(4,5-dihydro-4,4-dimethyloxazol-2yl)phenyl]borinic acid quinolin-8-yl ester (18)

In a manner as in Section 5.9.7.1, the titled compound was prepared from (3-chlorophenyl)[3-(4,5-dihydro-4,4-dimethyloxazol-2yl)phenyl]borinic acid and 8-hydroxyquinoline to afford a yellow crystalline solid. ESI-MS m/z 441 (M+H)⁺, C₂₆H₂₂B³⁵ClN₂O₂=440.

### 5.9.7.22 [3-(4,5-Dihydro-4,4-dimethyloxazol-2yl)phenyl](3-fluorophenyl)borinic acid quinolin-8-yl ester (19)

In a manner as in Section 5.9.7.1, the titled compound was prepared from [3-(4,5-dihydro-4,4-dimethyloxazol-2yl)(3-fluorophenyl)borinic acid and 8-hydroxyquinoline to afford a yellow crystalline solid. ESI-MS m/z 425 (M+H)⁺, C₂₆H₂₂BFN₂O₂=424.

### 5.9.7.23 Cyclopropyl[3-(4,5-dihydro-4,4-dimethyloxazol-2yl)phenyl]borinic acid quinolin-8-yl ester (20)

In a manner as in Section 5.9.7.1, the titled compound was prepared from cyclopropyl[3-(4,5-dihydro-4,4-dimethyloxazol-2yl)phenyl]borinic acid and 8-hydroxyquinoline to afford a yellow crystalline solid. ESI-MS m/z 371 (M+H)⁺, C₂₃H₂₃BN₂O₂=370.

### 5.9.7.24 [4-(4,5-Dihydro-4,4-dimethyloxazol-2yl)phenyl]vinylborinic acid quinolin-8-yl ester (21)

In a manner as in Section 5.9.7.1, the titled compound was prepared from [4-(4,5-dihydro-4,4-dimethyloxazol-2yl)vinyl borinic acid and 8-hydroxyquinoline to afford a yellow crystalline solid. ESI-MS m/z 357 (M+H)⁺, C₂₂H₂₁BN₂O₂=356.

### 5.9.7.25 (4-Cyanophenyl)(4-fluorophenyl)borinic acid quinolin-8-yl ester (22)

In a manner as in Section 5.9.7.1, the titled compound was prepared from (4-cyanophenyl)(4-fluorophenyl)borinic acid and 8-hydroxyquinoline to afford a yellow crystalline solid.

### 5.9.7.26 [4-(4,5-Dihydrooxazol-2-yl)phenyl]vinylborinic acid quinolin-8-yl ester (23)

In a manner as in Section 5.9.7.1, the titled compound was prepared from [4-(4,5-dihydrooxazol-2-yl)phenyl]vinylborinic acid 8-hydroxyquinoline to afford a yellow crystalline solid. ESI-MS m/z 329 (M+H)⁺, C₂₀H₁₇BN₂O₂=328.

### 5.9.7.27 (3-Cyano-4-fluorophenyl)vinylborinic acid quinolin-8-yl ester (24)

In a manner as in Section 5.9.7.1, the titled compound was prepared from (3-cyano-4-fluorophenyl)vinylborinic acid 8-hydroxyquinoline to afford a yellow crystalline solid. ESI-MS m/z 303 (M+H)⁺, C₁₈H₁₂BFN₂O=302.

### 5.9.7.28 (3-Chlorophenyl)(2-methylphenyl)borinic acid quinolin-8-yl ester (25)

In a manner as in Section 5.9.7.1, the titled compound was prepared from (3-chlorophenyl)(2-methylphenyl)borinic acid 8-hydroxyquinoline to afford a yellow crystalline solid. ESI-MS m/z 358 (M+H)⁺, C₂₂H₁₇B³⁵ClNO=357.

### 5.9.7.29 (3-Chlorophenyl)(4-cyanophenyl)borinic acid quinolin-8-yl ester (26)

In a manner as in Section 5.9.7.1, the titled compound was prepared from (3-chlorophenyl)(4-cyanophenyl)borinic acid 8-hydroxyquinoline to afford a yellow crystalline solid. ESI-MS m/z 369 (M+H)⁺, C₂₂H₁₄B³⁵ClN₂O=368.

### 5.9.7.30 (3-Chlorophenyl)(3,4-dimethoxyphenyl)borinic acid quinolin-8-yl ester (27)

In a manner as in Section 5.9.7.1, the titled compound was prepared from (3-chlorophenyl)(3,4-dimethoxyphenyl)borinic acid 8-hydroxyquinoline to afford a yellow crystalline solid. ESI-MS m/z 404 (M+H)⁺, C₂₃H₁₉B³⁵ClNO₃=403.

### 5.9.7.31 (4-Cyanophenyl)(3-fluorophenyl)borinic acid quinolin-8-yl ester (28)

In a manner as in Section 5.9.7.1, the titled compound was prepared from (4-cyanophenyl)(3-fluorophenyl)borinic acid 8-hydroxyquinoline to afford a yellow crystalline solid. ESI-MS m/z 353 (M+H)⁺, C₂₂H₁₄BFN₂O=352.

### 5.9.7.32 (3-Cyanophenyl)(3-fluorophenyl)borinic acid quinolin-8-yl ester (29)

In a manner as in Section 5.9.7.1, the titled compound was prepared from (3-cyanophenyl)(3-fluorophenyl)borinic acid 8-hydroxyquinoline to afford a yellow crystalline solid. ESI-MS m/z 353 (M+H)⁺, C₂₂H₁₄BFN₂O=352.

### 5.9.7.33 (2-Chlorophenyl)(3-fluorophenyl)borinic acid quinolin-8-yl ester (30)

In a manner as in Section 5.9.7.1, the titled compound was prepared from (2-chlorophenyl)(3-fluorophenyl)borinic acid 8-hydroxyquinoline to afford a yellow crystalline solid. ESI-MS m/z 362 (M+H)⁺, C₂₁H₁₄B³⁵ClFNO=361.

### 5.9.7.34 (4-Chloro-3-methylphenyl)(3-cyanophenyl)borinic acid quinolin-8-yl ester (31)

In a manner as in Section 5.9.7.1, the titled compound was prepared from (4-chloro-3-methylphenyl)(3-cyanophenyl)borinic acid 8-hydroxyquinoline to afford a yellow crystalline solid. ESI-MS m/z 383 (M+H)⁺, C₂₃H₁₆B³⁵ClN₂O=382.

### 5.9.7.35 (3-Cyanophenyl)(2,5-difluorophenyl)borinic acid quinolin-8-yl ester (32)

In a manner as in Section 5.9.7.1, the titled compound was prepared from (3-cyanophenyl)(2,5-difluorophenyl)borinic acid 8-hydroxyquinoline to afford a yellow crystalline solid. ESI-MS m/z 371 (M+H)⁺, C₂₂H₁₃BF₂N₂O=370.

### 5.9.7.36 (3-Cyanophenyl)(2-fluorophenyl)borinic acid quinolin-8-yl ester (33)

In a manner as in Section 5.9.7.1, the titled compound was prepared from (3-cyanophenyl)(2-fluorophenyl)borinic acid 8-hydroxyquinoline to afford a yellow crystalline solid. ESI-MS m/z 353 (M+H)⁺, C₂₂H₁₄BFN₂O=352.

### 5.9.7.37 (4-Chloro-3-methylphenyl)(4-cyanophenyl)borinic acid quinolin-8-yl ester (34)

In a manner as in Section 5.9.7.1, the titled compound was prepared from (4-chloro-3-methylphenyl)(4-cyanophenyl)borinic acid 8-hydroxyquinoline to afford a yellow crystalline solid. ESI-MS m/z 383 (M+H)⁺, C₂₃H₁₆B³⁵ClN₂O=382.

### 5.9.7.38 (4-Cyanophenyl)(2,5-difluorophenyl)borinic acid quinolin-8-yl ester (35)

In a manner as in Section 5.9.7.1, the titled compound was prepared from (4-cyanophenyl)(2,5-difluorophenyl)borinic acid 8-hydroxyquinoline to afford a yellow crystalline solid. ESI-MS m/z 371 (M+H)⁺, C₂₂H₁₃BF₂N₂O=370.

### 5.9.7.39 (2-Chlorophenyl)vinyl borinic acid quinolin-8-yl ester (36)

In a manner as in Section 5.9.7.1, the titled compound was prepared from (4-chlorophenyl)vinylborinic acid 8-hydroxyquinoline to afford a yellow crystalline solid. ESI-MS m/z 294 (M+H)⁺, C₁₇H₁₃B³⁵ClNO=293.

### 5.9.7.40 (4-Cyanophenyl)vinyl borinic acid quinolin-8-yl ester (38)

In a manner as in Section 5.9.7.1, the titled compound was prepared from (4-cyanophenyl)vinylborinic acid 8-hydroxyquinoline to afford a yellow crystalline solid. ESI-MS m/z 285 (M+H)⁺, C₁₈H₁₃B³⁵N₂O=284.

### 5.9.7.41 (3-Fluorophenyl)vinyl borinic acid quinolin-8-yl ester (39)

In a manner as in Section 5.9.7.1, the titled compound was prepared from (3-fluorophenyl)vinylborinic acid 8-hydroxyquinoline to afford a yellow crystalline solid. ESI-MS m/z 278 (M+H)⁺, C₁₇H₁₃B³⁵FNO=277.

### 5.9.7.42 (4-Chlorophenyl)(2,5-difluorophenyl)borinic acid quinolin-8-yl ester (40)

In a manner as in Section 5.9.7.1, the titled compound was prepared from (4-chlorophenyl)(2,5-difluorophenyl)borinic acid 8-hydroxyquinoline to afford a yellow crystalline solid. ESI-MS m/z 380 (M+H)⁺, C₂₁H₁₃B³⁵ClF₂NO=379.

### 5.9.7.43 (4-Chlorophenyl)(3-fluoro-4-methoxyphenyl)borinic acid quinolin-8-yl ester (41)

In a manner as in Section 5.9.7.1, the titled compound was prepared from (4-chlorophenyl)(3-fluoro-4-methoxyphenyl)borinic acid 8-hydroxyquinoline to afford a yellow crystalline solid. ESI-MS m/z 392 (M+H)⁺, C₂₂H₁₆B³⁵ClFNO₂=391.

### 5.9.7.44 (4-Chlorophenyl)(2,3-difluorophenyl)borinic acid quinolin-8-yl ester (42)

In a manner as in Section 5.9.7.1, the titled compound was prepared from (4-chlorophenyl)(2,3-difluorophenyl)borinic acid 8-hydroxyquinoline to afford a yellow crystalline solid. ESI-MS m/z 380 (M+H)⁺, C₂₁H₁₃B³⁵ClF₂NO=379.

### 5.9.7.45 (4-Chloro-2-fluorophenyl)(3-fluorophenyl)borinic acid quinolin-8-yl ester (43)

In a manner as in Section 5.9.7.1, the titled compound was prepared from (4-chloro-2-fluorophenyl)(3-fluorophenyl)borinic acid 8-hydroxyquinoline to afford a yellow crystalline solid. ESI-MS m/z 380 (M+H)⁺, C₂₁H₁₃B³⁵ClF₂NO=379.

### 5.9.7.46 (4-Chlorophenyl)(3,5-difluorophenyl)borinic acid quinolin-8-yl ester (44)

In a manner as in Section 5.9.7.1, the titled compound was prepared from (4-chlorophenyl)(3,5-difluorophenyl)borinic acid 8-hydroxyquinoline to afford a yellow crystalline solid. ESI-MS m/z 380 (M+H)⁺, C₂₁H₁₃B³⁵ClF₂NO=379.

### 5.9.7.47 (4-Chloro-3-methoxyphenyl)(3-fluorophenyl)borinic acid quinolin-8-yl ester (45)

In a manner as in Section 5.9.7.1, the titled compound was prepared from (4-chloro-3-methoxyphenyl)(3-fluorophenyl)borinic acid 8-hydroxyquinoline to afford a yellow crystalline solid. ESI-MS m/z 392 (M+H)⁺, C₂₂H₁₆B³⁵ClFNO₂=391.

### 5.9.7.48 (3,4-Dichlorophenyl)(3-fluorophenyl)borinic acid quinolin-8-yl ester (46)

In a manner as in Section 5.9.7.1, the titled compound was prepared from (3,4-dichlorophenyl)(3-fluorophenyl)borinic acid 8-hydroxyquinoline to afford a yellow crystalline solid. ESI-MS m/z 396 (M+H)⁺, C₂₁H₁₃B³⁵Cl₂FNO=395.

### 5.9.7.49 (4-Chlorophenyl)(3-fluoro-5-trifluoromethylphenyl)borinic acid quinolin-8-yl ester (47)

In a manner as in Section 5.9.7.1, the titled compound was prepared from (4-chlorophenyl)(3-fluoro-5-trifluoromethylphenyl)borinic acid 8-hydroxyquinoline to afford a yellow crystalline solid. ESI-MS m/z 430 (M+H)⁺, C₂₂H₁₃B³⁵ClF₄NO=429.

### 5.9.7.50 (3,5-Difluorophenyl)vinyl borinic acid quinolin-8-yl ester (48)

In a manner as in Section 5.9.7.1, the titled compound was prepared from (3,5-difluorophenyl)vinylborinic acid 8-hydroxyquinoline to afford a yellow crystalline solid. ESI-MS m/z 296 (M+H)⁺, C₁₇H₁₂B³⁵F₂NO=295.

### 5.9.8 Hydroxypicolinic Acid Derivatives

### 5.9.8.1 Bis(3-Chloro-4-methylphenyl)borinic acid 3-hydroxypicolinate ester (69)

Bis(3-chloro-4-methylphenyl)borinic acid (14.6 g) was dissolved in ethanol (120 mL) and heated to reflux. 3-Hydroxypicolinic acid (5.83 g) was added in portions to the hot solution. The reaction was stirred at reflux for 15 minutes after the addition of the last portion of 3-hydroxypicolinic acid was added and then cooled to room temperature. Reaction was concentrated by removal of some ethanol. Solid was removed by filtration. One recrystallization from ethanol afforded the title product as white crystals (13.4 g). MP=165.0-166.5° C. MS (ESI+): m/z=400 (M⁺+1).

In a preferred embodiment, the present invention includes anti-parasitic use of the compounds specifically recited herein, and pharmaceutically acceptable salts, hydrates, and solvates thereof; and compositions of any of these compounds where these comprise a pharmaceutically acceptable carrier.

The present invention also relates to a method for treating a microbial-caused disease in a patient afflicted therewith and/or preventing such infection in a patient at risk of becoming so-infected, comprising administering to said patient a therapeutically effective amount of any of the anti-parasitic compounds preferably one or more of those listed in Table 1.

In a preferred embodiment, the microbe is a parasite, wherein said parasite is a member selected from (but not limited to) the group consisting of Plasmodium falciparum, P. vivax, P. ovals P. malariae, P. berghei, Leishmania donovani, L. infantum, L. chagasi, L. mexicana, L. amazonensis, L. venezuelensis, L. tropica, L. major, L. minor, L. aethiopica, L. Biana braziliensis, L. (V.) guyanensis, L. (V.) panamensis, L. (V.) periviana. Trypanosoma brucei rhodesiense, T. brucei gambiense, T. cruzi, Giardia intestinalis, G. lamblia, Toxoplasma gondii, Entamoeba histolytica, Trichomonas vaginalis, Pneumocystis carinii, and Cryptosporidium parvum.

What is claimed:

1. A method for the treatment of a parasitic disease in an animal, comprising administering to such animal a therapeutically effective amount of a compound having the structure: 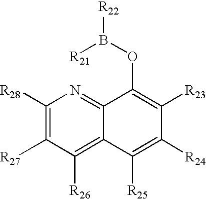
or its pharmaceutically acceptable salts, hydrates, or solvates, wherein:
R₂₁ and R₂₂ are selected independently from the group consisting of optionally substituted alkenyl, optionally substituted cycloalkyl, optionally substituted aryl, optionally substituted heteroaryl, and optionally substituted heterocyclic;
R₂₃-R₂₈ are selected independently from the group consisting of hydrogen, hydroxy, alkyl, alkoxy, halo, cyano, aryl, aralkyl, heteroaralkyl, heteroaryl, aryloxy, heterocycyloxy, heteroaryloxy, thio, alkylthio, arylthio, heteroarylthio, cycloalkyl, heterocycyl, cycloalkyloxy, formyl, carboxy, thioformyl, thiocarboxy, sulfonyl, alkylsulfonyl, arylsulfonyl, heteroarylsulfonyl, alkylsulfinyl, arylsulfinyl, heteroarylsulfinyl, amino, alkylamino, dialkylamino, arylamino, alkylsulfonylamino, arylsulfonylamino, and diarylamino, wherein each of the above-recited alkyl-, aryl-, and heteroaryl-containing moieties is optionally substituted.

2. The method of claim 1, wherein R₂₁ is optionally substituted alkenyl.

3. The method of claim 2, wherein R₂₁ is optionally substituted vinyl.

4. The method of claim 3, wherein R₂₂ is optionally substituted aryl or optionally substituted heteroaryl.

5. The method of claim 4, wherein R₂₂ is optionally substituted aryl.

6. The method of claim 5, wherein R₂₂ is phenyl substituted with at least one moiety selected from the group consisting of: cyano, halo, optionally substituted heteroaryl, and optionally substituted heterocyclic.

7. The method of claim 6, wherein said moiety is selected from the group consisting of: cyano, fluoro, chloro, 4,4-dimethyl-4,5-dihydrooxazol-2-yl, and 4,5-dihydrooxazol-2-yl.

8. The method of claim 7, wherein R₂₃-R₂₈ are selected independently from the group consisting of: hydrogen, hydroxy, alkoxy, thio, alkylthio, halo, alkyl, and cyano.

9. The method of claim 8, wherein R₂₃-R₂₇ are hydrogen.

10. The method of claim 9, wherein R₂₈ is hydroxy.

11. The method of claim 10, wherein said compound is selected from the group consisting of compounds 21, 23, 24, 36-39, and 48 of Table 1.

12. The method of claim 4, wherein R₂₂ is optionally substituted heteroaryl.

13. The method of claim 12, wherein R₂₂ is optionally substituted pyridyl.

14. The method of claim 13, wherein R₂₂ is 3-pyridyl.

15. The method of claim 13, wherein R₂₃-R₂₈ are selected independently from the group consisting of: hydrogen, hydroxy, alkoxy, thio, alkylthio, halo, alkyl and cyano.

16. The method of claim 15, wherein R₂₃-R₂₇ are hydrogen.

17. The method of claim 16, wherein R₂₈ is hydroxy.

18. The method of claim 1, wherein R₂₁ is optionally substituted cycloalkyl.

19. The method of claim 18, wherein R₂₁ is optionally substituted cyclopropyl.

20. The method of claim 19, wherein R₂₂ is optionally substituted aryl.

21. The method of claim 20, wherein R₂₂ is optionally substituted phenyl.

22. The method of claim 2 1, wherein R₂₂ is phenyl substituted with at least one moiety selected from the group consisting of: cyano, halo, optionally substituted heteroaryl, optionally substituted heterocyclic.

23. The method of claim 22, wherein said moiety is selected from the group consisting of: cyano, fluoro, chloro, 4,4-dimethyl-4,5-dihydrooxazol-2-yl, and 4,5 -dihydrooxazol-2-yl.

24. The method of claim 23, wherein R₂₃-R₂₈ are selected independently from the group consisting of: hydrogen, hydroxy, alkoxy, thio, alkylthio, halo, alkyl, and cyano.

25. The method of claim 24, wherein R₂₃-R₂₇ are hydrogen.

26. The method of claim 25, wherein R₂₈ is hydroxy.

27. The method of claim 1, wherein both R₂₁ and R₂₂ independently are optionally substituted aryl.

28. The method of claim 27, wherein both R₂₁ and R₂₂ independently are optionally substituted phenyl.

29. The method of claim 28, wherein both R₂₁ and R₂₂ independently are phenyl optionally substituted with at least one moiety selected from the group consisting of: halo, alkyl, alkoxy, cyano, and cycloheteroalkyl.

30. The method of claim 30, wherein R₂₃-R₂₈ are selected independently from the group consisting of: hydrogen, hydroxy, alkoxy, thio, alkylthio, halo, alkyl, and cyano.

31. The method of claim 32, wherein R₂₈ is hydroxy.

32. The method of claim 31, wherein R₂₃-R₂₇ are hydrogen.

33. The method of claim 32, wherein said compound is selected from the group consisting of compounds 10-15, 17-19, 20, 22, 25-35, and 40-47 of Table 1.

34. The method of claim 31, wherein R₂₅ is cyano and R₂₃, R₂₄, R₂₆, and R₂₇ are hydrogen.

35. The method of claim 34, wherein said compound is compound number 16 of Table 1.

36. The method of claim 1, wherein said parasitic disease is associated with a parasite selected from the group consisting of: Plasmodium falciparum, P. vivax, P. ovale, P. malariae, P. berghel, Leishmania donovani, L. infantum, L. chagasi, L. mexicana, L. amazonensis, L. venezuelensis, L. tropica, L. major, L. minor, L. aethiopica, L. Biana braziliensis, L. (V.) guyanensis, L. (V.) panamensis, L. (V.) periviana. Trypanosome brucei rhodesiense, I brucei gambiense, T. cruzi, Giardia intestinalis, G. lamblla, Toxoplasma gondii, Entamoeba histolytica, Trichomonas vaginalis, Pneumocystis carinii, and Crytosporidium parvum.

37. A method for the treatment of a parasitic disease in an animal, comprising administering to such animal a therapeutically effective amount of a compound having the structure 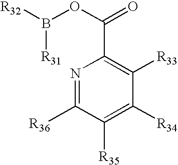
or its pharmaceutically acceptable salts, hydrates, or solvates, wherein:
R₃₁ and R₃₂ are selected independently from the group consisting of optionally substituted alkyl, optionally substituted aryl, aralkyl, and optionally substituted heteroaryl;
R₃₃-R₃₆ are selected from the group consisting of: hydrogen, arylcarbonyl, alkylcarbonyloxy, hydroxy, alkyloxy, amino, dialkylamino, diarylamino, alkylamino, arylamino, alkylsulfonylamino, arylsulfonylamino, carboxyalkyloxy, heterocycyloxy, carboxy, hydroxyalkyl, aminoalkyl, (alkylamino)alkyl, (dialkylamino)alkyl, alkyloxycarbonyl, carbamoyl, hydroxy, alkoxy, aryloxy, thio, alkylthio, arylthio, alkylsulfonyl, dialkylsulfamoyl, alkylsulfamoyl, sulfamoyl, sulfonyl, cyano, halo, nitro, alkylcarbamoyl alkylsulfinyl, arylsulfinyl, alkanoylamino, alkyl, sulfamoyloxy wherein each of the above-recited alkyl-, aryl-, and heteroaryl-containing moieties is optionally substituted; and
R₃₅ and R₃₆ together with the ring atoms to which they are attached form an optionally substituted aromatic ring.

38. The method of claim 37, wherein one of R₃₁ and R₃₂ is optionally substituted aryl.

39. The method of claim 38, wherein one of R₃₁ and R₃₂ is optionally substituted heteroaryl.

40. The method of claim 39, wherein said optionally substituted heteroaryl is optionally substituted pyridyl.

41. The method of claim 40, wherein one of R₃₁ and R₃₂ is optionally substituted phenyl.

42. The method of claim 38, wherein said optionally substituted phenyl is phenyl substituted by a moiety selected from the group consisting of: alkyl, cycloalkyl, aryl, substituted aryl, aralkyl, —(CH₂)ₖOH (where k=1, 2 or 3), —CH₂NH₂, —CH₂NH-alkyl, —CH₂N(alkyl)₂, —CO₂H, —CO₂alkyl, —CONH₂, —CONHalkyl, —CON(alkyl)₂, —OH, alkoxy, aryloxy, —SH, —S-alkyl, —S-aryl, —S(O)alkyl, —S(O)aryl, —SO₂alkyl, —SO₂N(alkyl)₂, —SO₂NHalkyl, —SO₂NH₂, —SO₃H, —SCF₃, —CN, halogen, —CF₃, —NO₂, amino, substituted amino, —NHSO₂alkyl, —OCH₂CH₂NH₂, —OCH₂CH₂NHalkyl, —OCH₂CH₂N(alkyl)₂, oxazolidin-2-yl, and alkyl substituted oxazolidin-2-yl.

43. The method of claim 38, wherein both R₃₁ and R₃₂ are optionally substituted aryl.

44. The method of claim 43, wherein both of R₃₁ and R₃₂ is optionally substituted phenyl.

45. The method of claim 44, wherein R₃₃ is hydrogen, hydroxy, alkoxy, or carboxy.

46. The method of claim 45, wherein said optionally substituted phenyl is phenyl substituted by a moiety selected from the group consisting of: alkyl, cycloalkyl, aryl, substituted aryl, aralkyl, —(CH₂)ₖOH (where k=1, 2 or 3), —CH₂NH₂, —CH₂NH-alkyl, —CH₂N(alkyl)₂, —CO₂H, —CO₂alkyl, —CONH₂, —CONHalkyl, —CON(alkyl)₂, —OH, alkoxy, aryloxy, —SH, —S-alkyl, —S-aryl, —S(O)alkyl, —S(O)aryl, —SO₂alkyl, —SO₂N(alkyl)₂, —SO₂NHalkyl, —SO₂NH₂, —SO₃H, —SCF₃, —CN, halogen, —CF₃, —NO₂, amino, substituted amino, —NHSO₂alkyl, —OCH₂CH₂NH₂, —OCH₂CH₂NHalkyl, —OCH₂CH₂N(alkyl)₂, oxazolidin-2-yl, and alkyl substituted oxazolidin-2-yl.

47. The method of claim 46, wherein R₃₃ is hydroxy or carboxy.

48. The method of claim 47, wherein said compound is a compound selected from Table 1.

49. The method of claim 47, wherein R₃₃ is hydroxy.

50. The method of claim 49, wherein said optionally substituted phenyl is phenyl substituted by a moiety selected from the group consisting of: hydrogen, halogen, and alkyl.

51. The method of claim 50, wherein said halogen is chloro.

52. The method of claim 51, wherein said alkyl is methyl.

53. The method of claim 52, wherein said compound is (bis(3-chloro-4-methylphenyl)boryloxy)(3-hydroxypyridin-2-yl)methanone.

54. The method of claim 53, wherein said compound is a solvate of said (bis(3-chloro-4-methylphenyl)boryloxy)(3-hydroxypyridin-2-yl)methanone.

55. The method of claim 53, wherein said compound is a hydrate of said (bis(3-chloro-4-methylphenyl)boryloxy)(3-hydroxypyridin-2-yl)methanone.

56. The method of claim 37, wherein said parasitic disease is associated with a parasite selected from the group consisting of: Plasmodium falciparum, P. vivax, P. ovale, P. malariae, P. berghel, Leishmania donovani, L. infantum, L. chagasi, L. mexicana, L. amazonensis, L. venezuelensis, L. tropica, L. major, L. minor, L. aethiopica, L. Biana braziliensis, L. (V.) guyanensis, L. (V.) panamensis, L. (V.) periviana. Trypanosome brucei rhodesiense, I brucei gambiense, T. cruzi, Giardia intestinalis, G. lamblla, Toxoplasma gondii, Entamoeba histolytica, Trichomonas vaginalis, Pneumocystis carinii, and Crytosporidium parvum.
